(19) United States
(12) Patent Application Publication
(10) Pub. No.: US 2008/0171002 A1
(43) Pub. Date: Jul. 17, 2008

(54) Products For Receptor Mediated Activation And Maturation Of Monocyte-Derived Dendritic Cells By A Phosphorylated Glucomannane Polysaccharide

(75) Inventors: Jose Antonio Matji Tuduri, Madrid (ES); Antonio F. Guerrero Gomez-Pamo, Madrid (ES); Jose Luis Alonso Lebrero, Madrid (ES); Garrett Lindemann, Big Horn, WY (US); Manuel Lopez Cabrera, Madrid (ES); Pedro Majano Rodriguez, Madrid (ES); Ricardo Moreno Otero, Madrid (ES); Diego Serrano Gomez, Madrid (ES); Angel Corbi Lopez, Madrid (ES); Samuel Martin Vilchez, Madrid (ES)

Correspondence Address: LATHROP & GAGE LC, 4845 PEARL EAST CIRCLE, SUITE 300, BOULDER, CO, 80301, US

(73) Assignee: GOURMETCEUTICALS, LLC, Big Horn, WY (US)

(21) Appl. No.: 11/781,135

(22) Filed: Jul. 20, 2007

Related U.S. Application Data
(60) Provisional application No. 60/832,316, filed on Jul. 20, 2006.

(51) Int. Cl. A61K 8/60; A61K 31/715; A61K 39/00; A61K 39/21; A61K 39/12; A61K 39/008; A61K 39/245; A61Q 1/00; A61Q 1/10; A61Q 19/00; A61Q 17/04; A61Q 9/00; A61Q 3/02; A61Q 1/06; A61P 37/02; A61K 39/29; A61K 39/02; A61K 39/108; A61K 39/04; A61K 39/395

(52) U.S. Cl. 424/59; 514/54; 424/184.1; 424/208.1; 424/204.1; 424/269.1; 424/230.1; 424/228.1; 424/218.1; 424/234.1; 424/259.1; 424/248.1; 424/133.1; 424/64; 424/61; 424/63; 424/65; 424/70.1

(57) ABSTRACT

Phosphorylated glucomannan polysaccharide compositions are shown to effectively enhance healthy immune function. Dosage forms including pills, sprays, functional foods and cosmetics may achieve this benefit while being essentially free of storage protein from nongerminated seeds of Ricinus communis.

### 1. RELATED APPLICATIONS

This application claims the benefit of priority to U.S. Provisional Patent Application No. 60/832,316, filed Jul. 20, 2006, which is incorporated herein by reference.

### 2. FIELD OF THE INVENTION

The field of this disclosure pertains to a phosphorylated glucomannan polysaccharide (PGPS) and use thereof in the activation and maturation of Monocyte-derived Dendritic Cells (DC). More particularly, the PGPS or a portion of the PGPS may bind to one or more receptors to activate a signal transduction pathway or repress a signal transduction pathway for improved immune response to infections and infectious diseases and restoration of a suppressed immune system.

### 3. DESCRIPTION OF THE RELATED ART

Dendritic cells (DC) are professional Antigen-Presenting Cells (APC) that links the innate and adaptive branches of the immune response through a capacity to recognize pathogen-associated structures and promote the initiation of T cell-dependent immunity¹. These cells exist in two functionally and phenotypically distinct states, which are termed immature and mature. Immature DCs localize in different tissues and organs, and act as “sentinels” that capture foreign antigen with high efficiency. Upon pathogen or “danger” challenge, DCs migrate to peripheral lymphoid organs, undergoing profound changes in phenotype and function, a process referred to as DC maturation. Different stimuli, such as pro-inflammatory cytokines (e.g. tumor necrosis factor α (TNF)-α and interleukin 1 (IL-1) and bacterial products such as lipopolysaccharide (LPS) may induce DC maturation in vivo and in vitro.

The biological process of DC maturation represents a key step in the initiation of adaptive immune responses. This process may be induced by various extra-cellular stimuli, including cytokines, bacterial products, and membrane-bound ligands^{3,2}. DC maturation is accompanied by a decrease of endocytic and phagocytic capacities, antigen uptake and processing, but results in increased antigen presentation^{3,4}. After maturation, DCs produce cytokines (e.g., IL-1, IL-10 and IL-12), which are essential for polarization of the T cell response towards Th1 or Th2⁵, as well as chemokines (e.g. monocyte chemoattractant protein (MCP)-1, macrophage-inflammatory protein (MIP)-1α, MIP-1β, IL-8), which favor lymphocyte recruitment and activation⁶. In addition, mature DCs express increased levels of surface antigens involved in T cell activation such as co-stimulatory molecules (e.g., CD54 and CD86) and MHC-I and MHC-II molecules, all of which results in enhanced antigen presentation and T cell proliferation-promoting ability^{3,4}.

DC maturation is fully dependent on NF-KB activation, which ultimately determines most of the phenotypic and functional parameters associated with this process^{7,8}. The three major mitogen-activated protein kinases (MAPK) signalling pathways in mammals, including p38 MAPK, extracellular signal-regulated kinases (ERK), and c-Jun N-terminal kinases (JNK), are activated in DC on maturation induced by LPS or TNF-α^{9,10}.

In the steady state, immature myeloid DC display a potent antigen uptake ability and contribute to the establishment of peripheral tolerance¹¹, whereas mature DC display a strong capacity for T cell stimulation and polarization of the immune response. Pathogen recognition by immature DC is carried out by a number of cell surface molecules named pathogen-associated molecular pattern (PAMP) receptors, which include the Toll-like receptor (TLR) family¹²and a large number of lectins and lectin-like molecules¹³, including the Dendritic Cell-Specific ICAM-3 Grabbing Nonintegrin (DC-SIGN, CD209) lectin. DC-SIGN is a type II membrane C-type lectin^{14,15,16} which recognizes a large array of viral, bacterial, fungal and parasite pathogens^{17,18,19,20,21,22,23,24,25,26} in a mannan- and Lewis oligosaccharides-dependent manner^{27,28}, and which mediates DC interactions with naïve T lymphocytes, endothelial cells and neutrophils via recognition of ICAM-3¹⁵, ICAM-2²⁹and Mac-1³⁰, respectively.

AM3 is the active agent of the drug Immunoferon®®^{35,36,31,32}, which has many therapeutic benefits, acts as an adjuvant after oral ingestion, and is not known to cause and side effects in clinical studies. AM3 is a glycoconjugate of natural origin composed of a phosphorylated glucomannan polysaccharide (PGPS) from Candida utilis and the storage protein from nongerminated seeds of Ricinus communis, RicC3³³and constitutes an immunoregulatory drug administered by oral route. In vivo, AM3 enhances lymphocyte proliferation, interleukin-2 production and NK activity³⁴. Immunoferon® functions as an adjuvant to hepatitis B revaccination in non-responder healthy persons^{35,36}, and partially rescues the defective natural killer and phagocytic activities seen in chronic obstructive pulmonary disease patients³⁷. Oral administration of AM3 increases IL-10 and reduces LPS-induced TNF-αt, IL-11 and i-NOS, thus acting as a modulator of the innate immune system by acting on peripheral blood mononuclear cells^{38,39}. In fact, AM3 triggers dendritic cell maturation and promotes the preferential release of IL-10 from mature human monocyte-derived dendritic cells.⁴⁰

Immunoferon® modulates several regulatory and effector functions of the immune system. In this context, AM3 acts on PBMC by promoting inflammatory mediators release that inhibits HBV replication in vitro⁴³, enhances cytotoxic activity of NK cells and increases lymphocyte proliferation, and IL-2 production in rodents⁴³. In addition, AM3 may induce an up-regulation of the 1 integrin ligand VCAM-1 and the β2 integrin counter-receptor ICAM-1 in human umbilical vein-derived endothelial cells⁴¹. AM3 also regulates corticoids in LPS-treated mice, constituting a potential mechanism to limit inflammation³⁸. Furthermore AM3 enhances the antibacterial immune response during systemic infection by Pneumocistis carinii⁴². Taken together, these results suggest that Immunoferon® functions as a modulator of the immune response by inducing a wide-range stimulation of immune cells, which collaborates in the control if endotoxic shock, viral and bacterial infections.

AM3 modulates, in vitro and in vivo, regulatory and effector functions of the immune system acting on peripheral blood mononuclear cells (PBMC)⁴³and enhancing lymphocyte proliferation, IL-2 production, and cytotoxic activity of Natural Killer (NK) cells³⁴. Furthermore, AM3 has been shown to reduce LPS-induced TNF-α³⁸, and inducible Nitric Oxide Synthase (iNOS) expression³⁹and it elevates serum levels of corticoids in untreated animals and enhances corticoids expression in LPS-challenged mice³⁸. Immunoferon® is administrated for non-specific activation of the immune system and to prevent recurrent infections. However, a mechanism of action for the active principle AM3 as well as identification of the receptors that it binds to and the specific cells that are activated remains unexplained and unidentified, respectively.

Structurally, AM3 is a glucomannan that is isolated from the cell wall of Candida utilis. Mannan-type polysaccharides from plant, bacterial and fungal sources have been described to have immunomodulatory effects, although their macrophage activating potential appears to be weaker than that of β-glucans. As one example, Acemannan, a polydispersed 1-(1,4)-linked mannan used for the treatment of fibrosarcoma, wounds and burns, is an immunostimulant which causes macrophage activation⁴⁴. In the case of Mycobacterium, lipoarabinomannans (LAM) affect a wide array of biological functions⁴⁵. However, subtle differences in LAM structure result in opposite functional properties. Whereas mannosyl cap-containing LAMs (ManLAM) are anti-inflammatory molecules and inhibit TNF-α and IL-12 production by mononuclear phagocytes, phosphoinositol-capped LAMs (PILAMs), which lack manno-oligosaccharide caps, are pro-inflammatory molecules capable of stimulating the production of TNF-α and IL-12. These differential effects of ManLAM and PILAMs underline the correlation between the presence of mannan and their immunomodulatory effect⁴⁶.

Only mannosylated LAMs have been shown to be recognized by DC-SIGN on the surface of human dendritic cells¹⁵. Therefore, the ability of PGPS to bind DC-SIGN maybe in agreement with its ability to reduce LPS-induced TNF-α, IL-11 and i-NOS production by human mononuclear cells^{27,28}.

A recently described ability of A. fumigatus cell wall galactomannan is to inhibit, not only the capture of fungal conidia by DC-SIGN, but also the DC-SIGN/ICAM-3 interaction²⁶. Interestingly, the structure of PGPS and the A. fumigatus galactomannan differ from that of the Lewis X (Galβ1-4(Fucα1-3)GlcNAc) and pseudo-LewisY (Fucα1-3Galβ1-4(Fucα1-3)GlcNAc) determinants, which are the DC-SIGN glycolipids ligands in Schistosoma mansoni cercariae⁴⁶.

The flexibility in the sugar-recognition activity of DC-SIGN is thought to be the basis for its ability to recognize a large array of pathogens, all of which may target DC-SIGN as a means to evade the immune response⁴⁷. Upon ligation by pathogenic or endogenous ligands, DC-SIGN is rapidly internalized from the cell surface and found in intracellular vesicles, where DC-SIGN mediates antigen delivery into endocytic/lysosomal compartments for subsequent loading of MHC molecules and effective antigen presentation^{48,49}. For this reason DC-SIGN has been proposed as an efficient target for antibody-mediated delivery of T cell epitopes in vaccine development⁵⁰. In fact, monoclonal antibodies against DC-SIGN are extremely potent at inducing antigen-specific CD4+ T cell proliferation⁵¹, and humanized anti-DC-SIGN antibodies have been shown to be effective inducers of naïve and recall T cell responses⁵⁰.

Lectin receptors on dendritic cells trigger intracellular signals which modulate those arising from TLR molecules⁵¹. As a representative example, ligation of the β-glucan receptor Dectin-1 synergizes with TLR2 to induce TNF-α and IL-12, and promotes IL-10 synthesis through recruitment of the Syk kinase⁵². In the case of DC-SIGN, recognition of mycobacterial lipoarabinomannan leads to production of IL-10 and suppression of dendritic cell activity^{24,53}. Moreover, the simultaneous presence of LPS and anti-DC-SIGN cross-linking antibodies results in enhanced production of IL-10 by human monocyte-derived dendritic cells (MDDC), without significantly affecting the release of IL-12p70. Induction of maturation that takes place upon addition of PGPS onto MDDC, which results in IL-10 producing mature MDDC with an enhanced ability to stimulate T cell proliferation⁴⁰.

### SUMMARY

The present instrumentalities overcome the problem of glucomannan immunological activity, specifically Immunoferon®, and advance the art by providing a mechanism of action for the maturation and activation of Dendritic Cells (DC) by a glucomannan composition. Knowledge of this mechanism of action permits the administration of glucomannan compositions for treatments or delivery that may be performed in a controllable and repeatable way. Notable instances of such compositions include PGPS, and especially AM3. Accordingly, AM3 may now be utilized to treat immunological diseases and increase pathogen recognition by human, mammalian and higher animal dendritic cells.

By way of example, the results below show that AM3 binds specifically to the DC-SIGN protein, preventing the attachment of pathogens and altering the functionality of the receptor. Knowledge of this binding mechanism facilitates improved treatment modalities, such as first diagnosing an infection with a pathogen that binds to DC-SIGN. Additionally, the binding of PGPS or AM3 to the DC-SIGN molecule directly influences the pathogen recognition by the dendritic cells, and so knowledge of this binding mechanism permits improved uses of AM3 or PGPS as an adjuvant. In this second example, PGPS induces the maturation of DC cells and induces an enhancement of the immune system, for example, when co-administered with an antibiotic, a vaccine, or a nutrient that supports immune function.

In one aspect, a composition for enhancing immune function may contain. A mannan polysaccharide complex carbohydrate having a capacity to bind with DC-SIGN. The mannan polysaccharide complex carbohydrate is present in an effective amount for immunomodulation of the immune system, which is minimally from 1 to 5 mg per kg of body weight of a target animal. The effective amount may be greater depending upon the a disease of condition that is being addressed and may for example, be 20 mg per kg, 40 mg per kg, 100 mg per kg or more. The composition may also contain a co-active agent for stimulating an immune response. The co-active agent is combined with the mannan polysaccharide complex for increased benefit of the immune response by immunomodulation from the mannan polysaccharide complex.

The mannan polysaccharide complex carbohydrate is preferably a phosphorylated glucomannan polysaccharide, such as may be derived from Candida utilis, which is optionally digested into shorter chain components representing up to about 225% complete hydrolysis. Alternatively, the mannan polysaccharide complex carbohydrate may be derived from fungus or plants.

In one aspect, the co-active agent may include a vaccine. The vaccine may be formulated to provide immunity against a pathogen that binds with DC-SIGN. Pathogens that bind with DC sign include, for example, HIV-1, Ebola virus, Leishmania pifanoi, Cytomegalovirus, Hepatitis C, Dengue virus, Helicobacter pylori, Klebsiella pneumonae, Mycobacterium tuberculosis, Schistosoma mansoni, and Coxiella burnetii.

In one aspect, the co-active agent may include a treating agent for infectious disease. This may include an antibiotic, such as those in the class of aminoglycosides including amikacin, gentamicin, kanamycin, neomycin, netilmicin, streptomycin, and tobramycin; carbacephems including loracarbef, ertapenem, imipenem/cilastatin, and meropenem; cephalosporins including cefadroxil, cefazolin, cephalexin; cefaclor, cefamandole, cefoxitin, cefprozil, cefuroxime, cefixime, cefdinir, cefditoren, cefoperazone, cefotaxime, claforan, cefpodoxime, ceftazidime ceftibuten, ceftizoxime, ceftriaxone, cefepime, and maxipime; glycopeptides including teicoplanin and vancomycin; macrolides including azithromycin, clarithromycin, dirithromycin, eythromycin, and troleandomycin; monobactam including aztreonam; penicillins including amoxicillin, ampicillin, azlocillin, carbenicillin, cloxacillin, dicloxacillin, flucloxacillin, mezlocillin, nafcillin, penicillin, piperacillin, and ticarcillin; polypeptides including bacitracin, colistin, and polymyxin B; quinolones including ciprofloxacin, enoxacin, gatifloxacin, levofloxacin, lomefloxacin, moxifloxacin, norfloxacin, ofloxacin, and trovafloxacin; sulfonamides incluing mafenide, prontosil, sulfacetamide, sulfamethizole, sulfanilimide, sulfasalazine, sulfisoxazole, trimethoprim, and trimethoprim-sulfamethoxazole; tetracyclines including demeclocycline, doxycycline, minocycline, oxytetracycline, and tetracycline; and others including chloramphenicol, clindamycin, ethambutol, fosfomycin, furazolidone, isoniazid, linezolid, metronidazole, nitrofurantoin, pyrazinamide, quinupristin/dalfopristin, rifampin and spectinomycin.

In one aspect, the co-active agent may include a nutrient that provides support for beneficial immune response. This nutrient may be a vitamin, especially vitamins A, B-6, biotin, C, D, and/or E. This nutrient may be a mineral, especially Cu, Fe, Se, Cr, Co, Zn, and/or salts thereof.

The composition may be formulated for oral, nasal, injectable, or topical administration. The composition may be formulated as a food product, at lest including food products other than a capsule or tablet, such as a human or animal feed, a candy or confection, a snack bar, a gel, a cosmetic, or a lotion. The formulation is provided by mixing the composition with a conventional food product. The composition may also be provided as a capsule or tablet.

The composition is used by delivering the same to an animal where internal action works the composition to produce the immune response and the immunomodulation. The method of use optionally but preferably includes a step of diagnosing the animal with an infectious condition that is caused by a pathogen where the animal is in need of treatment. In some embodiments, the pathogenesis of the pathogen includes binding to DC-SIGN. Such pathogens may include fungi, parasites, viruses, bacteria, and prions. By way of example, pathogens that bind to DC-SIGN include at least the species consisting of Candida, Aspergillus, Mycobacterium, Pneumocistis, Schistosoma and Leishmania, as well as viruses including virus as Ebola, HIV, or Hepatitis C. Specific organisms that bind to DC-SIGN include HIV-1, Ebola virus, Leishmania pifanoi, Cytomegalovirus, Hepatitis C, Dengue virus, Helicobacter pylori, Klebsiella pneumonae, Mycobacterium tuberculosis, Schistosoma mansoni, and Coxiella burnetii.

The mannan polysaccharide complex carbohydrate may also bind with a pattern recognition molecule including lectins, toll like receptors or both. In one example, the toll like receptor may be receptor-4 protein (TLR-4). In these instances, the method of use may also include a step of diagnosing the animal with an infectious condition in need of treatment where the infectious condition results from a pathogen that binds to the pattern recognition molecule.

The animal may be a human animal, or a non-human animal such as a food animal or pet animal. Specific examples of non-human animals include non-human primates, birds or poultry, equine species, bovine species, reptiles, and fish. The working of the composition may induce the maturation of dendritic cells, cause internalization of the receptor/carbohydrate complex, increases the rate and capture of an antigen or a mixture of antigens, increase the rate and capture of an epitope or a mixture of epitopes, increase the rate and capture of a hapten or a mixture of haptens, and/or increasing the rate and capture of a hapten or a mixture comprised of antigens, epitopes and haptens.

The composition may be used subsequent to diagnosis of condition that is in need of treatment by use of the composition. Such conditions as these include inflammatory disease or conditions with inflammatory components, a suppressed immune system, conditions that are caused by pathogens, cancer, infection, neurological disease, cardiac disease, blood disease, skeletal disease, disease of the muscle tissue, and/or disease caused by a prion. The diagnosis may also be for a primary condition that has secondary results included in the aforementioned conditions, such as diabetes.

Accordingly, the co-active agent may includes an antibiotic, antifungal, anti-viral, anti-prion, humanized monoclonal antibodies, humanized protein receptor with Fc immunoglobulin structure, anti-inflammatory, steroid, or anti-cancer drug that is complements the diagnosis to provide treatment for the condition. The composition may also be used in combination with administering radiation ultraviolet or near visible therapy, as well as radiation therapy. The composition may be used in combination with administering chemotherapy. In other aspects, DC cells may be isolated and treated ex vivo with the mannan polysaccharide complex carbohydrate, then later injected into the animal.

### BRIEF DESCRIPTION OF THE DRAWINGS

FIG. 1 shows the results of a binding study that confirms PGPS inhibits the binding of Candida albicans to monocyte-derived dendritic cells.

FIG. 2 shows the results of a binding study that confirms PGPS inhibits the binding of Aspergillus fumigatus to monocyte-derived dendritic cells.

FIG. 3 shows the results of a binding study that confirms PGPS inhibits the binding of Candida albicans and Aspergillus fumigatus to K562-CD209 cells.

FIG. 4 shows the results of a binding study that confirms PGPS (1F-S) inhibits the binding of Leishmania pifanoi amastigotes to monocyte-derived dendritic cells and K562-CD209 cells.

FIG. 5 shows the results of a binding study that confirms PGPS (1F-S) inhibits the DC-SIGN recognition of HIV-1 gp120.

FIG. 6 shows the results of a binding study that confirms PGPS (1F-S) inhibits the DC-SIGN-dependant adhesive functions as binding of MDDC and K562-CD209 to ICAM-3.

FIG. 7 shows the results of a binding study that confirms PGPS (1F-S) inhibits the DC-SIGN-dependant homotypic aggregation of K562-CD209 cells in a dose-dependant manner.

FIG. 8 shows the results of a binding study that confirms PGPS (1F-S) promotes DC-SIGN internalization in monocyte-derived dendritic cells.

FIG. 9 shows results from ID saturation transfer difference NMR experiments confirming that PGPS interacts with DC-SIGN.

FIG. 10 shows flow cytometry results confirming that PGPS up-regulates cell surface molecules of human MDDC.

FIG. 11 shows flow cytometry results confirming that PGPS diminishes FITC-dextran uptake on MDDC.

FIG. 12 shows ELISA assay results that confirm PGPS induces IL-12p70 and IL-10 production by human DC cells.

FIG. 13 shows ELISA results that confirm PGPS increases the proliferation of allogenic T cells and IFN-γ production

FIG. 14 shows the results of RNAse protection assay results confirming that PGPS increases localization of p65/reaA and promotes IkBα degradation and p38 MARK phosphorylation.

FIG. 15 shows the results of immunofluorsence studies that confirm PGPS induces chemokine and chemokine receptors mRNA expression by human immature DC cells.

FIG. 16 shows the results of a transfection study that confirms PGPS induces TLR-4-mediated NFκB activation.

FIG. 17 presents the results of biometric analysis to identify sequences that function as analogues to human DC-SIGN through use of the NCBI non-redundant sequence database.

FIGS. 18 and 19 include sequence listing reports from bioinformatic databases.

### DETAILED DESCRIPTION

The immunomodulatory action of polysaccharides has been known for decades, but their precise molecular, cellular and physiological mechanism has not been fully determined. Since PGPS binds with DC-SIGN, it also inhibits the binding and capture of fungal (e.g., Candida, Aspergillus) and parasite (e.g., Leishmania) pathogens by human monocyte-derived dendritic cells in a dose-dependent manner. This effect is mediated through interaction of PGPS with the DC-SIGN pathogen-attachment factor. PGPS also prevents the activity of DC-SIGN as a mediator of cell adhesion by impairing the DC-SIGN interaction with ICAM-3. Results indicate that PGPS directly influences pathogen recognition of dendritic cells by interacting with DC-SIGN on the cell surface, and suggest that the adjuvant and immunomodulatory action of PGPS are mediated, at least partly, by altering the functional capabilities of DC-SIGN.

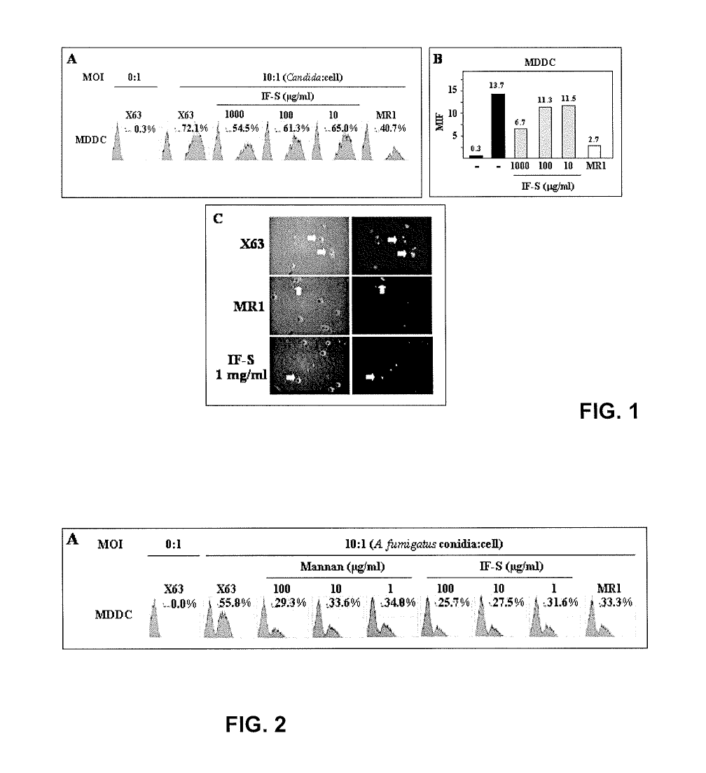

The state of the art is advanced by the insight gained into the immunoregulatory actions mediated by Immunoferon® and demonstrates that AM3 induces phenotypical, and functional changes in human MDDC. AM3 induces a significant up-regulation of DC maturation markers including MHC class II, co-stimulatory and adhesion molecules (HLA-DR, CD86, CD83, CD54) in MDDC (see FIG. 1). The endocytic activity of AM3-treated MDDC with respect to the internalization of FITC-dextran was decreased compared to immature MDDC (see FIG. 2). Also, AM3 augmented MDDC capacity to promote the proliferation of allogenic T cells (see FIG. 4).

AM3 also promotes the generation of functionally active, mature DC cells, and this implicates a number of downstream immune responses. T cells priming requires the activation of DC, which are activated by recognition of characteristic pattern of pathogens as well as by inflammatory cytokines. Depending on the stimulus encountered, DC can induce Th1, Th2, regulators T cells or unpolarized T cells responses⁵⁴. IL-12 plays a central role as a link between the innate and adaptive immune systems. Thus, IL-12 induces and promotes NK and T cells to generate IFN-γ and lytic activity. In addition, IL-12 polarizes the immune system toward a primary T helper cell type 1 (Th1) response⁵⁵. IL-10 is a pleiotropic cytokine produced by DC, T cells, and macrophages with anti-inflammatory and immunosuppressive properties polarizing toward a primary T helper cell type 2 (Th2) responses⁵⁶.

Strikingly, AM3 also induces increased expression of the cytokine IL-10. These apparently conflicting results agree with previous reports, demonstrating that LPS potentiates IL-10 and IL-12 expression by DC⁵⁷, up-regulating IL-10 to limit IL-12 production and controlling the inflammatory response58. In fact, the lower IL-12/IL-10 production ratio induced by AM3 compared to LPS suggests that the drug mimics, only partially, the pro-inflammatory pathways induced by LPS. The data herein demonstrate that AM3 induces IFN-γ secretion (pro-Th1 cytokine) without affecting significantly IL-4 production (pro-Th2). This data suggests that, at least under the experimental conditions analyzed, AM3 promotes Th1 T lymphocyte polarization.

The function of DC cells is intimately connected to their capacity to migrate. DC precursors are recruited from the bloodstream into tissues either constitutively or in response to chemotactic signals. Once in tissues DC may be activated by inflammatory cytokines such as TNF-α and IL-1 or by bacterial products such as LPS. These stimuli induce DC to mature and migrate via afferent lymph to the T cell areas of secondary lymphoid organs, where they acquire the capacity to stimulate naïve T cells⁶. AM3 up-regulates mRNA expression of chemokines such as IL-8, MCP-1, MIP-1α and MIP-1β in MDDC. These chemokines are involved in the recruitment of wide array of cell types including T cells, monocytes, neutrophils, and immature DC⁵⁹. Furthermore, DC maturation results in a switch in chemokine receptor expression with down-regulation of receptors for inflammatory chemokines, including CCR1, and up-regulation of receptors for chemokines, such as CCR7 and CXCR4⁵⁹. The significance of CCR7 up-regulation is of key relevance for homing of mature DC, since the CCR7 ligands are produced in secondary lymphoid organs⁶. Similar results were observed when MDDC were treated with LPS.

Recent reports have shown that LPS and TNF-α, two potent DC maturation factors, induced NF-κB activation and phosphorylation of p38 in MDDC^{7,9}. Moreover, the p38 MAPK pathway has been shown to contribute to NF-κB-mediated transactivation. The results herein demonstrate that NF-κB and one member of MAPK pathways, p38, were activated when immature human DC were exposed to AM3, suggesting a role of these pathways in the maturation promoted by AM3.

The mechanism through which AM3 stimulates immature MDDC was subsequently analyzed by determining potential cell surface receptors for AM3. Recognition of pathogens is mediated by a set of germline encoded receptors that recognize conserved molecular patterns shared by large groups of microorganisms. Toll-like receptors (TLRs) play an essential role in the recognition of microbial components and endogenous ligands induced during innate immune responses¹². Two members of the TLRs, TLR2 and TLR4, both implicated in bacterial component recognition, trigger similar cellular transduction pathways, promoting MAPK and NF-κB activation⁶⁰. LPS interaction with TLR-4 preferentially activates p38MAPK, whereas TLR-2 ligands, peptidoglycan and bacterial lipoproteins, preferentially activate ERK 1/2^{61,62}. Results indicate that, similar to LPS, AM3 is able to interact with TLR4, but fails to with TLR-2 expressing cells, resulting in the activation of NF-κB. Altogether these results suggest that AM3 might be a TLR4 partial-agonist, and that this interaction could be accounting, at least in part, of the effects of AM3 on MDDC maturation.

Immunoferon® is administrated orally and is transported into the intestinal lumen, where it can be delivered to the DC localized in the mucosa⁶³. Additionally, the data below suggest that common signaling pathways are activated by AM3 and LPS. In this regard, the adjuvant activity of bacterial products is important not only for antibacterial responses induced by peripheral DC but also for vaccine development. However, LPS is excluded because of its high toxicity, as it is one of the main causative agents of septic shock in humans⁶⁴. Therefore, the ability of AM3 to mimic signaling pathways induced by LPS and its lack of systemic toxicity⁶⁵suggests its potential employment as an adjuvant in vaccination protocols in which mature DC, could be used as antigen carriers

From this point of view, the ability of PGPS to block pathogen binding to DC-SIGN indicates that it constitutes a useful tool to prevent the access of clinically relevant pathogens to dendritic cells, whose regulated migratory behavior contributes to pathogen dissemination.

The previously reported adjuvant activity of Immunoferon® is directly linked to two other relevant aspects of DC-SIGN, namely the function as an endocytic receptor and the signalling capability in dendritic cells. Based on these findings, it can be anticipated that PGPS binding to DC-SIGN might contribute to enhance the rate of antigen capture and internalization by dendritic cells, thus explaining its' previously described adjuvant activity. The identification of DC-SIGN as a specific receptor for PGPS will certainly help to understand the molecular mechanisms for this adjuvant activity, and might allow the generation of PGPS-derived molecules with improved adjuvant efficacy.

The PGPS-binding ability of DC-SIGN and its intracellular signalling capability also explain some of the previously described effects of AM3.

As a consequence, the immunomodulatory activities of PGPS are explained by an ability to ligate DC-SIGN on the surface of dendritic cells and macrophages. In this manner, PGPS promotes intracellular signals favoring production of IL-10, and modulate the signals arising from other pathogen recognition receptors.

The following examples teach by way of example, and not by limitation.

### EXAMPLE 1

### Phosphorylated Glucomannan Polysaccharide (PGPS) Prevents Pathogen Binding by Monocyte-Derived Dendritic Cells Through Inhibition of DC-Sign Recognition Capabilities

### Materials and Methods

Glucomannan polysaccharide preparation. —The phosphorylated glucomannan polysaccharide from the cell wall of Candida utilis (hereafter termed PGPS) was obtained according to the methods described in patents P9900408 (Spain) and PCT/ES99/00338. Endotoxin contamination of the PGPS preparation was assayed with the Test Pyrogent plus kit (Bio Whittaker, Rockland, Me.), which has a detection threshold of 0.0625 UI/ml. Endotoxin was not detected even at concentrations 1000-times higher than those used in functional experiments.

Generation of monocyte-derived dendritic cells (MDDC) and cell culture. —Human peripheral blood mononuclear cells (PBMC) were isolated from buffy coats from normal donors over a Lymphoprep (Nycomed Pharma, Oslo, Norway) gradient according to standard procedures. Monocytes were purified from PBMC by magnetic cell sorting using CD14 microbeads (Miltenyi Biotech, Bergisch Gladbach, Germany). To generate monocyte-derived dendritic cells (MDDC), CD14+ cells (>95% monocytes) were cultured at 0.5-1×10⁶cells/ml in RPMI with 10% fetal calf serum (FCS), 25 mM HEPES and 2 mM glutamine (complete medium), at 37° C. in a humidified atmosphere with 5% CO2. Differentiation into immature MDDC was accomplished by the addition of GM-CSF (Immunotools) and IL-4 (Immunotools), both at 1000 U/ml. Medium was replaced and new cytokines added every 2 days. After 5-to-7 days cells were in suspension and exhibited the phenotypic and functional characteristics of immature dendritic cells. K562 cells stably transfected with DC-SIGN (K562-CD209) have been previously described and were cultured in complete medium containing 300 μg/ml G418. Mock-transfected K562 cells (stably transfected with an empty pcDNA3.1-plasmid) were used as control.

Flow cytometry and antibodies. —Cells were collected, washed in ice-cold PBS and resuspended in 100 μl of complete medium containing 50 μg/ml of human IgG and incubated for 15 min at 4° C. to prevent binding through the Fc portion of the antibodies. Then, 100 μl of a solution containing 10 μg/ml of the indicated monoclonal antibodies were added and incubated for 30 min on ice. After 3 washing steps in PBS, cells were resuspended in 100 μl of complete medium containing FITC-labeled F(ab′)2 rabbit anti-mouse IgG, kept on ice for 30 min, washed and resuspended in 200 μl of PBS for flow cytometry. Monoclonal antibodies included anti-CD209 (DC-SIGN, MR-1) and anti-Mannose Receptor 2.1D10 (anti-CD206, Mannose Receptor, generously provided by Dr. S. J. Sung, Department of Internal Medicine, University of Virginia Health Sciences Center, Charlottesville, Va.). Cells were also incubated with isotype-matched control antibodies and the supernantant of the non-producing myeloma P3X63 (X63) to determine the basal level of fluorescence. Flow cytometry analysis was performed with an EPICS-CS (Coulter Cientifica, Madrid, Spain) using log amplifiers.

DC-SIGN internalization assays. —MDDC were washed, resuspended in complete medium (2.5×10⁵cells per time point) and incubated with PGPS at distinct concentrations for one hour at 4° C. to prevent internalization. After extensive washing, cells were placed at 37° C. to allow internalization to occur. At the indicated time points, internalization was stopped by adding 4 volumes (200 μl) of cold PBS and cells were immediately placed at 4° C. Then cells were subjected to DC-SIGN and Mannose Receptor cell surface detection by flow cytometry using the MR-1 and 2.1D10 antibodies and a 1:100 dilution of an FITC-labeled goat anti-mouse antibody (Serotec). All incubations were done in the presence of 50 mg/ml of human IgG to prevent binding through the Fc portion of the antibodies. Flow cytometry analysis was performed with an EPICS-CS (Coulter Cientifica, Madrid, Spain) using log amplifiers.

Aspergillus fumigatus and Candida albicans binding assays. —Conidia from A. fumigatus or C. albicans were washed twice, resuspended and incubated in PBS containing 0.1 mg/ml FITC for 1 hr at room temperature. Fungi were then extensively washed and either used immediately or stored at −20° C. until use. Cells (MDDC or K562 transfectants) were washed, resuspended in complete medium (3×10⁵/well) and left untreated or pretreated for 20 minutes at room temperature with anti-DC-SIGN antibody (MR1) or PGPS or S. cerevisiae mannan at distinct concentrations. Then cells were incubated with FITC-labeled fungi at various ratios and the binding allowed to proceed for 30 min at room temperature. After extensive washing to eliminate unbound fungi, cells were fixed with 2% paraformaldehyde for 1 hr at 4° C., washed and analyzed on a Coulter EPICS-CS (Coulter Cientifica, Madrid, Spain).

Leishmania amastigotes-binding assay. —MDDC or K562 transfectants were washed in PBS 1 mM EDTA, resuspended in complete medium and aliquoted in 24-well plates (2×10⁵cells/well). 5,6-carboxyfluorescein succinimidyl ester (CFSE)-labeled Leishmania pifanoi amastigotes were added onto the cells at a 5:1 (amastigotes:cell) ratio, and incubated at room temperature for 30 minutes. Afterwards, cells were fixed (2% paraformaldehyde in PBS) for 1 h at room temperature, and analyzed by flow cytometry using an EPICS-CS (Coulter Cientifica, Madrid, Spain). For inhibition assays, cells were washed with PBS, 1 mM EDTA and preincubated for 10 min at room temperature with either the anti-DC-SIGN MR-1 antibody (1.2 μg/ml) or distinct concentrations of PGPS in complete medium before parasite addition.

DC-SIGN-dependent adhesion assays. —Adhesion to ICAM-3- or polysaccharide-coated plates. —DC-SIGN-dependent adhesion was evaluated using ICAM-3/Fc (kindly provided by Dr. Donald Staunton, ICOS Corporation, Bothwell, Wash.) or PGPS as ligands. 96-well microtiter EIA II-Linbro plates were coated overnight at 4° C. with ICAM-3/Fc at 3 mg/ml in 100 mM NaHCO3 pH 8.8, or PGPS at distinct concentrations (0.05-50 μg/ml) in PBS, and the remaining sites were blocked with 0.4% BSA for 2 h at 37° C. Cells were labeled in complete medium with the fluorescent dye 2′, 7′-bis-(2-carboxyethyl)-5-(and-6)-carboxyfluorescein acetoxymethyl ester (Molecular Probes) and then preincubated for 20 min at 37° C. in RPMI 1640 medium containing 0.4% BSA and containing or not the function-blocking antibody MR1 against DC-SIGN (MR1), Mannan or distinct PGPS concentrations. Cells were allowed to adhere to each well for 15 min at 37° C. Unbound cells were removed by three washes with 0.5% BSA in RPMI 1640 medium, and adherent cells were quantified using a fluorescence analyzer.

DC-SIGN-dependent aggregation. —K562-CD209 cells were washed, maintained in PBS 1 mM EDTA for 5 minutes, and resuspended in complete medium at 2×10⁵cells/mil. 500 μl of this cell suspension was then seeded onto tissue-culture plates containing either 500 μl of complete medium or 500 μl of complete medium containing A. fumigatus Galactomannan (100 μg/ml), a blocking antibody against DC-SIGN (MR-1 at 10 μg/ml) or PGPS at distinct concentrations. Homotypic aggregation was allowed to proceed for 20 minutes, and cells were analyzed by flow cytometry and photographed.

NMR experinments. —All the experiments were recorded on a Bruker 500 MHz at 298 K. A basic Saturation Transfer Difference (STD) sequence was used, with on-resonance frequency variable between 6.8 ppm or 1.3 ppm.⁶⁶. The success of the STD experiments depends on the kinetics of the dissociation process and the molar ratio of ligand versus receptor^{67,68}. Off-resonance frequency was maintained fixed at 100 ppm. A train of 40 gaussian-shaped pulses of 50 ms each was employed, with a total saturation time of the protein envelope of 2 s. On and off-resonance scans were alternated and recorded separately. In order to perform the STD experiments, 3.6×106 cells (K562-CD209 or mock-transfected K562) were washed, dissolved in 250 μl of deuterated PBS pH 7.3 (containing 1 mM of CaCl₂previously exchanged with D2O) and mixed with 2.5 mg of the PGPS preparation dissolved in 250 μl of the same deuterated PBS⁷¹???. Typical NMR tube volume was 450 μl. The estimated concentration of DC-SIGN in the NMR tube was approximately 0.1 μM. Robust data were obtained with 2×32 scans, corresponding to a total experimental time of 5 minutes. In order to determine the biological stability of the employed cells, these were checked by optical microscopy before and after the NMR experiments and cell viability was evaluated by Trypan Blue exclusion.

### Results

PGPS inhibits C. albicans binding to human dendritic cells. —Given the origin of the PGPS, analysis was performed to determine whether PGPS could affect the capture of pathogenic strains of Candida by dendritic cells. To that end human monocyte-derived dendritic cells were incubated with FITC-labeled C. albicans spores at a 10:1 ratio and determined the effect of a wide range of concentrations of PGPS. PGPS inhibited the binding of C. albicans to dendritic cells in a dose dependent manner as determine by either flow cytometry (FIG. 1A) or fluorescence microscopy (FIG. 1C). The inhibitory action of PGPS was similar to that of a function-blocking anti-DCSIGN monoclonal antibody (FIG. 1A). Furthermore, the presence of PGPS not only reduced the number of dendritic cells with bound C. albicans, but also diminished the number of fungi bound per cell, as evidenced by the inhibition of the MIF of the cell population (FIG. 1B). Therefore, the polysaccharide component of PGPS is capable of preventing the binding of C. albicans to human dendritic cells, which has been previously shown to be a DC-SIGN-dependent activity²⁵.

To determine whether PGPS exerted a similar inhibitory action with other pathogenic fungi, binding assays were performed with Aspergillus fumigatus conidia. As shown in FIG. 2, PGPS dose-dependently inhibited the binding of A. fumigatus conidia to human monocyte-derived dendritic cells, and the degree of inhibition was comparable to that obtained with equivalent concentrations of S. cervisiae mannan. Moreover, and like in the case of C. albicans, the inhibitory effect of PGPS was similar to the inhibition observed in the presence of an anti-DC-SIGN antibody (FIG. 2).

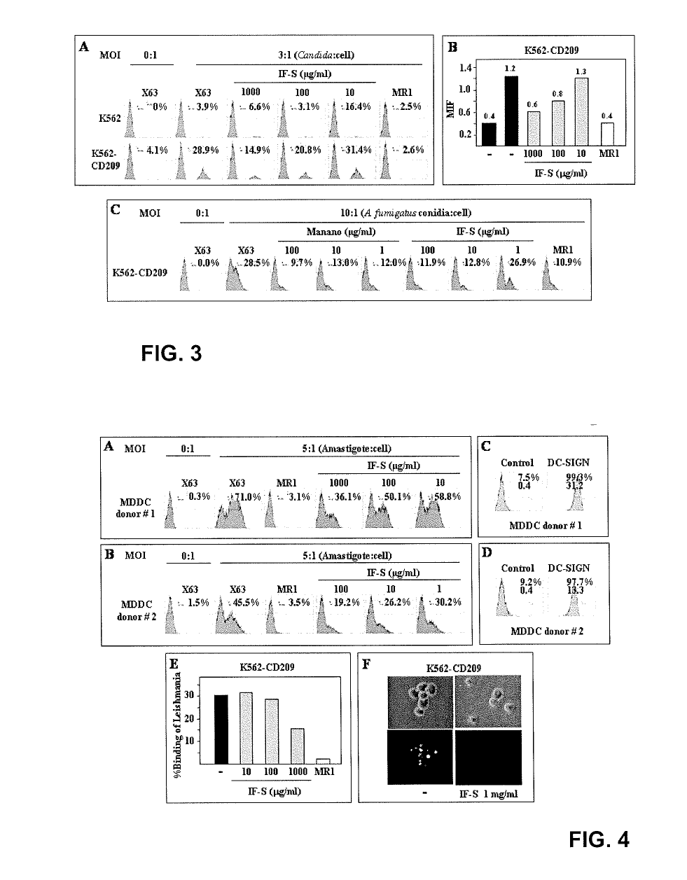

The polysaccharide component of PGPS specifically inhibits DC-SIGN-dependent pathogen-binding activities of human dendritic cells. —The binding and capture of C. albicans and A. fumigatus by human monocyte-derived dendritic cells is mediated, at least partially, by the C-type lectin DC-SIGN^{25,26}. Based on the above results, and to determine whether PGPS was directly affecting DC-SIGN dependent functions, binding assays were performed with FITC-labeled fungi on K562 cells stably transfected with DC-SIGN. PGPS also inhibited the binding of C. albicans to DC-SIGN transfectants in a dose-dependent manner, reaching 50% inhibition at the maximal concentration assayed (FIG. 3A). Like in the case of dendritic cells, the inhibitory action could be seen upon determination of both the number of cells with bound fungi and the number of fungi bound per cell (FIG. 3B). A. fumigatus binding to DC-SIGN transfectants was also inhibited in the presence of PGPS, which reduced fungal binding to less than 50% at 100 μg/ml (FIG. 3C). In fact, PGPS inhibited fungal binding as effectively as mannan, a well-known inhibitor of all DC-SIGN-dependent functions (FIG. 3C). Altogether, these results demonstrated that PGPS inhibits fungal binding to human dendritic cells by preventing the recognition ability of DC-SIGN, thus indicating that DC-SIGN directly binds PGPS.

Besides mediating fungal binding and capture, DC-SIGN functions as the major cell surface receptor for Leishmania in human monocyte-derived dendritic cells^{22,66}. To further evidence the ability of PGPS to impair DC-SIGN-dependent functions, we determined its effect on Leishmania binding by both dendritic cells and DC-SIGN transfectants. PGPS inhibited the binding of Leishmania amastigotes to dendritic cells from two distinct donors at concentrations as low as 10 μg/ml, with inhibitions of at least 50% at the highest concentrations assayed (FIG. 4A,B). As expected, an anti-DC-SIGN antibody completely blocked Leishmania attachment to human MDDC (FIG. 4A,B). Interestingly, the inhibitory effect of PGPS showed a certain level of donor-dependent variability which appeared to correlate with the level of expression of DC-SIGN: MDDC with lower DC-SIGN expression levels were more susceptible to the inhibitory action of PGPS (compare inhibition in FIGS. 4A and B with the DC-SIGN expression data in FIG. 4C,D), again supporting the involvement of DC-SIGN in the inhibitory action of PGPS.

The importance of DC-SIGN for the PGPS inhibitory effects were also demonstrated in Leishmania binding to K562-CD209 cells. As shown in FIG. 4E, PGPS was capable of inhibiting the attachment of Leishmania amastigotes to DC-SIGN transfectants, reaching 50% inhibition at 1 mg/ml. The effects of PGPS could be also observed in fluorescence microscopy experiments: the binding of Leishmania to K562-CD209 cells result in the formation of cellular aggregates caused by the simultaneous binding of several cells to a single Leishmania amastigote, whereas the presence of PGPS prevented the formation of such aggregates (FIG. 4F). Altogether these set of results confirmed that PGPS impairs pathogen recognition by dendritic cells by virtue of its ability to inhibit DC-SIGN-dependent functions.

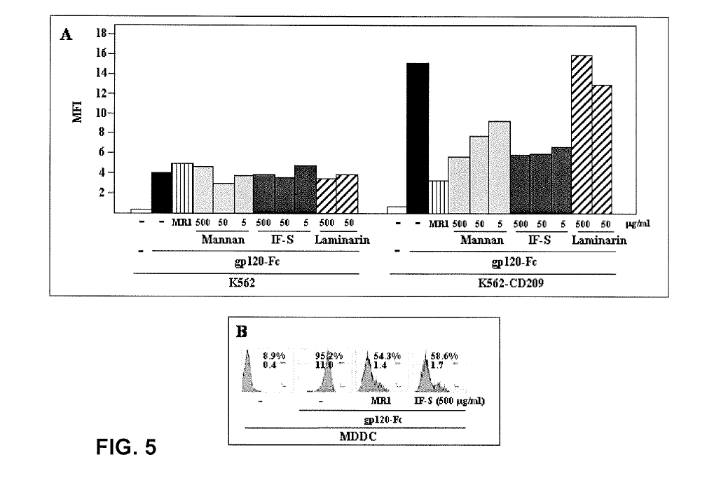

PGPS inhibits recognition of HIV-1 gp120 by DC-SIGN. —The ability of PGPS to block the pathogen recognition ability of DC-SIGN prompted us to determine whether PGPS could also inhibit the recognition of HIV-1 gp120 by DC-SIGN, which mediates HIV-1 attachment to DC-SIGN expressing cells⁶⁷. PGPS was capable of inhibiting the binding of gp120 to K562-CD209 cells in a dose-dependent manner, and the degree of inhibition caused by PGPS was similar to that caused by S. cerevisiae mannan (FIG. 5A). As a control, laminarin, which is a β-glucan polysaccharide ligand of Dectin-1⁶⁸, had no effect on gp120 binding to DC-SIGN (FIG. 5A). More importantly, binding of gp120 to MDDC was greatly inhibited by the anti-DC-SIGN MR-1 antibody, and a similar inhibitory effect was observed in the presence of PGPS (FIG. 5B). Therefore, PGPS inhibits the binding of HIV-1 gp120 to DC-SIGN on either transfectants or human monocyte-derived dendritic cells.

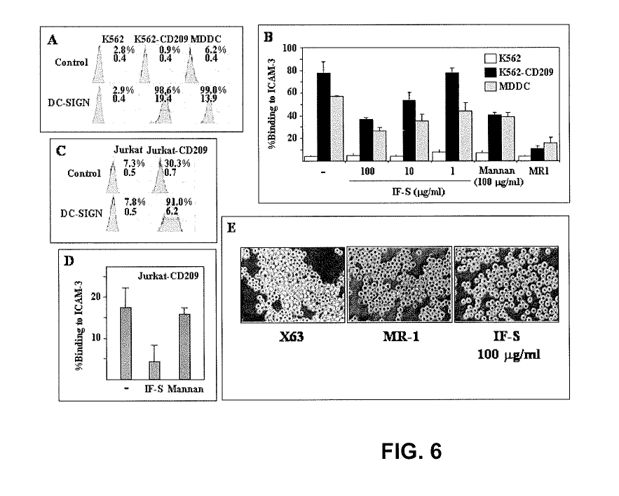

PGPS inhibits the ICAM3-binding ability of DC-SIGN. —The above results indicated that PGPS impairs DC-SIGN-dependent pathogen-binding capabilities of dendritic cells. However, DC-SIGN also functions as a cell adhesion molecule, mediating dendritic cell adhesion to lymphocytes, endothelial cells and neutrophils by interactions with ICAM-3, ICAM-2 or Mac-1, respectively^{15,69,70}. In an effort to evaluate whether PGPS might also influence DC-SIGN dependent adhesive functions, MDDC and K562-CD209 cells were allowed to bind to ICAM-3 in adhesion assays in the absence or presence of distinct concentrations of PGPS. Both cell types exhibited similar levels of cell surface DC-SIGN (FIG. 6A) and their binding to ICAM-3 was reduced by 50% in the presence of either PGPS or S. cerevisiae mannan at 100 μg/ml (FIG. 6B). The interference of PGPS on cell binding to ICAM-3 was also evident using Jurkat cells stably transfected with DC-SIGN, although in this cell type PGPS exhibited a higher inhibitory effect than mannan (FIG. 6C, D). These results indicate that PGPS inhibits the DC-SIGN-dependent adhesion of dendritic cells and DC-SIGN+ cells to ICAM-3.

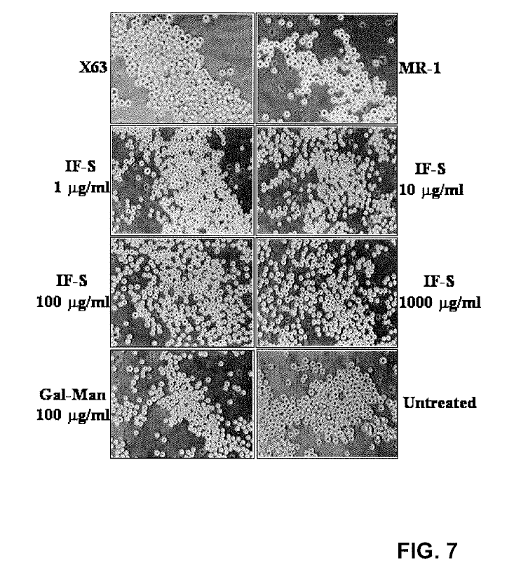

Growth of K562-CD209 cells result in the formation of homotypic aggregates whose formation can be abrogated by blocking anti-DC-SIGN antibodies and depends on the interaction of DC-SIGN with an unknown ligand⁷¹. As shown in FIG. 6E, the presence of PGPS at 100 μg/ml completely prevented the formation of homotypic aggregates of K562-CD209 cells. In fact, PGPS was even more effective than the MR-1 anti-DC-SIGN antibody in blocking the DC-SIGN-dependent homotypic aggregation (FIG. 6E). A dose-response analysis revealed that PGPS at 10 μg/ml also prevented the DC-SIGN-dependent homotypic aggregation, whereas PGPS at 1 μg/ml inhibited aggregation to a similar extent as the MR-1 blocking antibody (FIG. 7). Therefore, PGPS prevents the DC-SIGN-dependent cell adhesion to ICAM-3 as well as the homotypic aggregation of K562-CD209 cells.

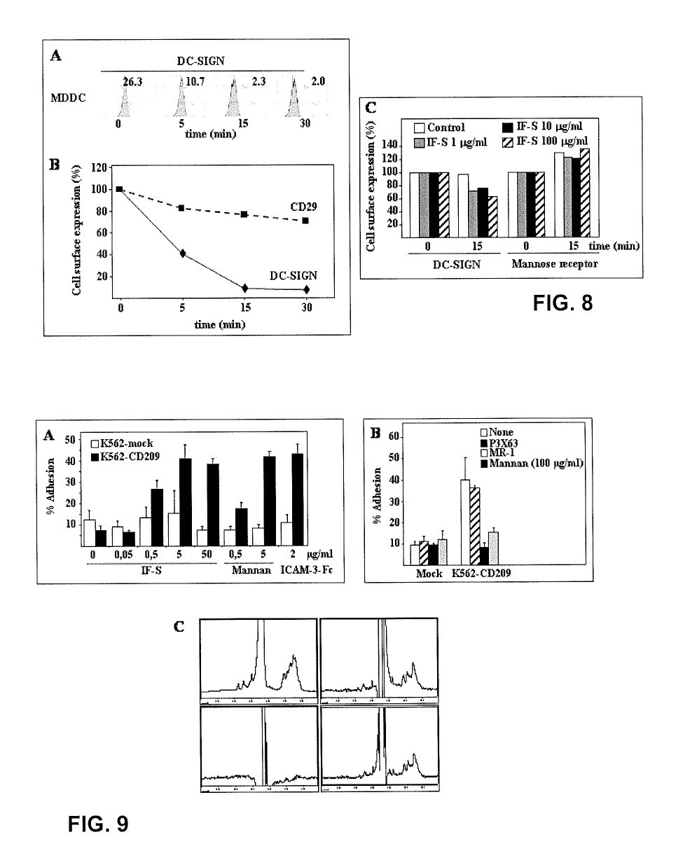

The presence of PGPS promotes DC-SIGN internalization and results in diminished cell surface expression of DC-SIGN on human dendritic cells. —All the previous results indicated that PGPS inhibits DC-SIGN functional activities on either dendritic cells or transfected cells. Given the ability of DC-SIGN to internalize its cognate ligands in dendritic cells (references), we reasoned that the direct interaction between PGPS and DC-SIGN should result in diminished expression of DC-SIGN. Therefore, to obtain evidences of such a direct interaction, human monocyte-derived dendritic cells were incubated with PGPS at 37° C. and the cell surface expression level of DC-SIGN was determined after distinct time points. In the presence of PGPS the expression of DC-SIGN was dramatically reduced, with 50% of the molecules being internalized only after 5 minutes, and further reduction of cell surface expression at later time points (FIG. 8A, B). By contrast no significant change in the expression of the CD29 was detected at any time point (FIG. 8B). More importantly, no alteration in the expression of the mannose receptor was detected in the presence of PGPS (FIG. 8C). Therefore, and although DC-SIGN and the Mannose receptor display similar adhesive and internalization capabilities, the presence of the polysaccharide moiety of PGPS only promotes internalization of DC-SIGN in dendritic cells.

PGPS binds and directly contacts DC-SIGN on the cell surface. —All the previous experiments revealed that PGPS inhibits DC-SIGN-dependent adhesive functions and, therefore, indirectly suggested that PGPS is directly recognized by DC-SIGN. To directly test this suggestion, adhesion assays were performed with DC-SIGN transfectants on PGPS, using ICAM-3 and mannan as positive controls. K562-CD209 cells bound to PGPS in a dose-dependent manner, reaching maximal adhesion at 5 mg/ml (FIG. 9A). Moreover, K562-CD209 cell binding to PGPS could be prevented in the presence of either mannan or the MR-1 blocking monoclonal antibody against DC-SIGN (FIG. 9B). Therefore DC-SIGN mediates cell binding to PGPS.

To more definitively demonstrate the DC-SIGN/PGPS interaction we used an alternative strategy. It has been recently demonstrated that STD NMR experiments might be used, in favorable cases, to detected ligand binding to receptors expressed at the surface of living cells⁷². STD experiments provide NMR signals that permit to deduce the existence of magnetization transfer between protons belonging to both the ligand and the receptor. This phenomenon can only take place if there is an interaction between both entities, whereas no STD signal is detected in the absence of interaction. The experimental approach is very robust and can be used for very high ligand/receptor molar ratios⁷³. Since we have previously demonstrated the feasibility of this STD-based methodology to the study of cell surface DC-SIGN-glucomannan interactions⁷⁴, we assayed whether a direct interaction between DC-SIGN and the PGPS polysaccharide could be demonstrated. To that end, 1D saturation transfer difference NMR experiments were performed with PGPS and either K562-CD209 (DC-SIGN⁺) or Mock transfected K562 cells (DC-SIGN−). The regular 1D 1H NMR spectrum of PGPS in the presence of K562 cells is shown in FIG. 9C (upper left panel). This regular NMR spectrum was identical for PGPS either alone or in the presence of either K562-CD209 or mock-transfected K562 cells. The STD control spectrum of the PGPS confirmed that the on-resonance irradiation (7 ppm, aromatic region) did not affect the polysaccharide signals, and identical results were obtained employing saturation at −0.3 ppm (aliphatic side chain region) (data not shown). When control NMR data of mock-transfected K562 dendritic cells were taken in the presence of PGPS, no polysaccharide signal was evidenced in the difference spectrum (FIG. 9C, lower left panel), indicating that PGPS does not interact with mock-transfected K562 cells. In contrast, the STD spectrum of the mixture of PGPS with the DC-SIGN containing K562-CD209 upon irradiation at either—0.3 ppm (FIG. 8C, upper right panel) or 7 ppm (FIG. 9C, lower right panel) unambiguously revealed the presence of polysaccharide signals. Therefore, irradiation at the aromatic or aliphatic regions protons of the DC-SIGN receptor protein expressed on living cells produces transfer of magnetization to the polysaccharide protons of PGPS, thus demonstrating the interaction of the PGPS polysaccharide with the cell surface. Since DC-SIGN-negative K562 cells do not produce any NMR signal, it can be safely concluded that the observed STD signals are due to the interaction of PGPS with the DC-SIGN receptor on the cell surface.

### EXAMPLE 2

### Phosphorylated Glucomannan Polysaccharide (PGPS) Induces Functional Human Dendritic Cell Maturation

### Material and Methods

### Reagents

LPS from Escherichia coli serotype (055:B5) was purchased from Sigma Chemical Co. (St. Louis, Mo.). PGPS was prepared according to the methods described in patents P9900408 (Spain) and PCT/ES99/00338. Briefly, the phosphorylated glucomannan polysaccharide from the cell wall of Candida utilis and a storage protein from Ricinus communis seeds (12 kD), were combined in a 5:1 (w/w) polysaccharide/protein proportion as described³⁸. PGPS was assayed for bacterial endotoxin employing the Test Pyrogent plus kit (Bio Whittaker, Rockland, Me.), which has a detection threshold of 0.0625 UI/ml. Endotoxin was not detected even at concentrations 1000-times higher than those used in functional experiments.

Generation and immunophenotyping of MDDC

PBMC were purified from healthy donors by Ficoll density centrifugation (Histopaque-1077; Sigma Diagnostics). CD14⁺ cells were purified by positive selection using anti-CD14⁺ microbeads in conjunction with the MiniMACS system by following the manufacturer's instructions (Miltenyi Biotec, Auburn, Calif.). The CD14⁺ cells were cultured at 1×10⁶cells per 1 ml RPMI 1640 (Life Technologies, Merelbeke, Belgium) containing 10% fetal calf serum and 20 μg/ml gentamicin in 6-well plates (Costar, Cambridge, Mass.) supplemented with granulocyte macrophage-colony stimulating factor (GM-CSF; 1000 U/ml) and IL-4 (1000 U/ml) (Preprotech, Rocky Hill, N.J.). Fresh medium containing GM-CSF and IL-4 was added every 2-3 days. Human MDDC were used routinely at day 5-6 of culture. All the experiments were carried-out in RPMI containing 10% fetal calf serum except those to determine MAPK's levels where immature MMDC were cultured with medium containing 0.5% FCS 16 hours before treatments. Cell viability was estimated by using propidium iodide or Trypan Blue to rule out the possibility that some of the effect PGPS were due to toxicity.

The following antibodies were employed: HLA-DR (DR), CD54/ICAM-1 (Hu5/3), ICAM-3 (TP1/24), kindly provided by Dr. Francisco Sanchez-Madrid (Hospital Universitario de la Princesa, Madrid, Spain) and FITC-conjugated antibodies against CD86 and CD83, purchased from Caltag Laboratories (Burlingame, Calif.). Immunostaining of unstimulated or stimulated MDDC was performed as follows: 10⁵cells were incubated with the antibodies described above or their isotype-matched controls for 20 min at 4° C. and rinsed twice with ice-chilled PBS. When required, a secondary FITC-conjugated goat anti-rabbit Ab (Dako, Glostrup, Denmark) was employed. Finally, fluorescence intensity was measured using a FACSCalibur® flow cytometer (BD Biosciences).

FITC-dextran uptake by MDDC

To measure particle uptake by MDDC, cells (5×10⁴) were resuspended in 100 μl of PBS containing 1% human serum and incubated with FITC-dextran (0.1 mg/ml) (Sigma Co.) at either 37° C. or 4° C. for 30 min. The process was stopped by addition of 2 ml ice-cold PBS containing 1% human serum. Cells were washed three times with ice-cold PBS and analyzed by flow cytometry.

### Measurement of Cytokines Levels

The IL-12 p70, and IL-10 in the culture supernatants from MDDC were assayed with enzyme-linked immunosorbent assay (ELISA) kits (R&D Systems, Minneapolis, Minn.), following the manufacturer's instructions. IFN-γ and IL-4 were determined in 5 days co-culture supernatants of MMDC/T lymphocytes supernatants by standard ELISA immunoassays (Pierce Endogen, Rockford, Ill.), according to the manufacturer's protocols.

### Allogeneic T-Lymphocyte Proliferation Induced by MDDC

PBMC were obtained from healthy adults as described above. Allogeneic CD3⁺ T lymphocytes were isolated by immunomagnetic negative selection using Pan T cell Isolation Kit II human (Miltenyi Biotec) according to the manufacturer's guide. For T-lymphocyte proliferation experiments, 2×10⁵T cells were stimulated in a 96-well plate with 0.1, 0.5, 2.5, or 10×10³radiated (1.5 Gy/min for 10 minutes) allogeneic MDDC matured under the different culture conditions. After a 5-day incubation period, tritiumthymidine was added (0.037 MBq/well) during the last 16 hours of co-culture and thymidine incorporation was determined to assess the level of T-cell proliferation.

### Determination of NF-KB Activity

MDDC were plated on gelatin-coated coverlips, allowed to settle for 30 minutes and treated with LPS (0.1 μg/ml) or PGPS (1 μg/ml) for different time points (15 minutes to 4 hours). Subcellular localization of NF-κB was analyzed by immunofluororescence with a specific polyclonal antibody against the NF-κB family member RelA/p65 (Santa Cruz Biotechnology, Santa Cruz, Calif.). Briefly, cells were fixed with 4% paraformaldehyde in PBS for 10 minutes at room temperature, permeabilized 5 minutes in PBS containing 0.1% Triton X-100, blocked 30 minutes at 37° C. with BSA (Boehringer Mannheim), and incubated 1 hour with an 1:1000 dilution of the antibody. Cells were then washed 3 times in PBS and labelled with a Cy3-conjugated rabbit anti-goat antibody (Jackson, Calif.). Coverlips were mounted with fluorescent mounting medium (Dako) and representative fields were photographed on a Nikkon Eclipse E-800 microscope (Nikkon, Melville, N.J.).

MAPKs and IkB [symbol] Expression

MDDC were left un-stimulated or stimulated with LPS or PGPS for different times ranging for 5 min to 24 h. Protein levels measurements were done by Western blot using specific polyclonal antibodies against ERK 1/2, p38, phospho-ERK 1/2, and phospho-p38 (Cell Signaling, Beverly, Mass.), and IkB [symbol] Santa Cruz Biotechnology) as previously described⁷⁵.

Chemokines and Chemokines Receptor mRNA Expression

DCs were stimulated for 12 h with LPS or PGPS, and total RNA was extracted from cultured cells using the ULTRASPEC RNA isolation system (Biotecx Laboratories, Houston Tex.). The expression of human chemokines and chemokine receptors mRNAs were determined by RNAse protection assays. Multi-probe template set hCK5 (containing DNA templates for Ltn, RANTES, IP-10, MIP-1α, MIP-1β, MCP-1, IL-8, I-309, L32, GAPDH), hCR5 (CCR1, CCR3, CCR4, CCR5, CCR8, CCR2a+b, CCR2a, CCR2b, L32, GAPDH) and hCR6 (CXCR1, CXCR2, CXCR3, CXCR4, CXCR5/BLR-1, CCR7/BLR-2, V28/CX3CR1, L32, GAPDH) were purchased from PharMingen (Pharmingen, San Diego Calif.) and experiments were carried-out according to the manufacturer's protocols.

Determination of PGPS/TLR 2 and TLR 4 interaction

HEK293-TLR4 and HEK293-TLR2 cells (kindly provided by Dr. Douglas T. Golenbock, University of Massachusetts, Worcester, Mass.), stably transfected with human TLR4 and TLR2, respectively, were transiently transfected with 1 μgr of the reporter vector KB-Luc, containing a trimer of the H-2 K^{b}gene NF-κB motif upstream of the luciferase reporter gene⁷⁶using Superfect (Quiagen, Valencia, Calif.) according to the manufacturer's recommendations. After 24 hours, cells were trypsinized and plated in 96 wells plate (10³cells/well) for 12 hours. After incubation with different PGPS concentrations for 6 hours, cells were harvested, lysed and luciferase activity determined using the Luciferase Assay System Kit (Promega, Valencia, Calif.). As positive controls HEK293-TLR4 cells were stimulated with LPS and HEK293-TLR2 cells with Pam3Cys (Invivogen, San Diego, Calif.).

### Reproducibility of the Data Between Different Donors and PGPS Batches

MDDC were generated from PBMC from healthy donor. At least three independent experiments were performed from each set of experiments. It is important to note that although the values showed varied slightly between donors, the overall tendency remained unchanged. Two different batches of PGPS were used for reported experiments with comparable results.

### Results

PGPS Induces Human MDDC Maturation Parameters.

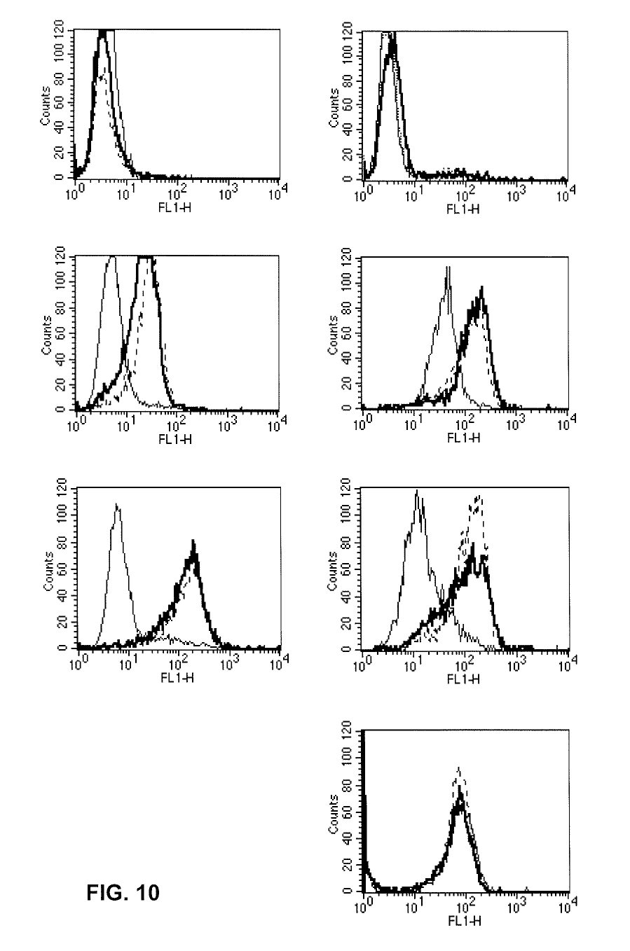

Immature DC comparable to those found in nonlymphoid tissues can be generated by culturing human peripheral blood monocytes in medium supplemented with GM-CSF and IL-4⁷⁷. To assess the effect of PGPS in MDDC, we incubated human MDDC with medium alone or different doses of PGPS (0, 1, 1, 10 μg/ml) for 24 h, and then assessed the expression of a selected panel of DC markers, including the MHC class II molecule HLA-DR, CD86/B7-2, a co-stimulatory molecule required for T cell activation, CD83, a specific marker of mature DC, and the adhesion molecules CD54/ICAM-1 and ICAM-3 (FIG. 10A). PGPS induced a marked dose-dependent up-regulation expression of all these markers, except for ICAM-3. Data shown in FIG. 1 were obtained from MDDC stimulated with 1 μg/ml of PGPS, a concentration that promoted phenotypical changes without affecting MDDC viability. The phenotypic changes induced by PGPS were comparable to those elicited by LPS (0.1 μg/ml), a positive control that promote MDDC maturation (FIG. 10), thus suggesting that PGPS induces MDDC maturation.

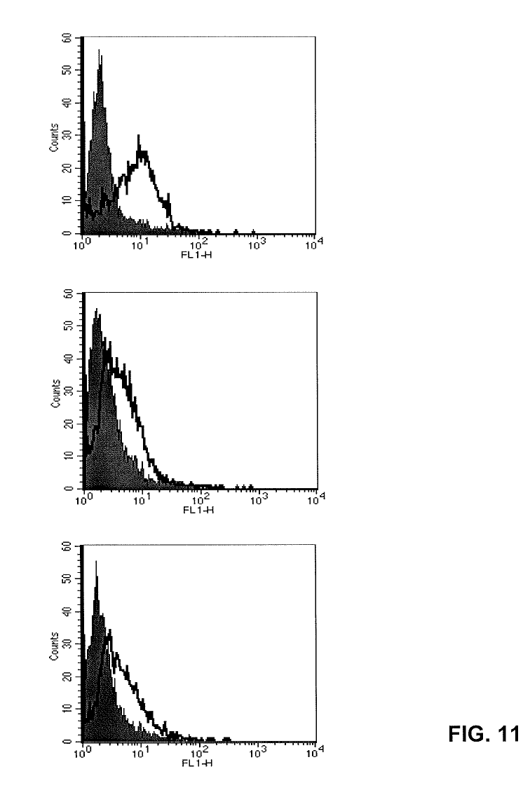

To further confirm the maturation-inducing effect PGPS on MDDC, we measured FITC-dextran uptake, which is reduced during DC maturation. Consistent with the effect on MDDC phenotype changes, PGPS-stimulated MDDC also exhibited a lower capacity of particle uptake when compared to un-stimulated MDDC (FIG. 11), further supporting the notion that PGPS promotes DC maturation.

### PGPS Enhances Bioactive IL-12 and IL-10 Production by MDDC

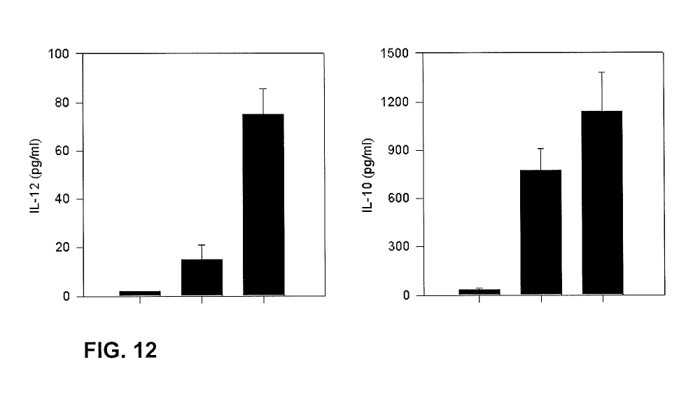

To investigate whether the phenotypic switch induced by PGPS correlates with a change in cytokine expression, we analyzed the expression of IL-10 and IL-12p70 whose expression is induced during MDDC maturation in response to LPS⁵⁷. As shown in FIG. 3A, PGPS significantly induced the expression of IL-12 p70, although to a lesser extent than LPS. However, both PGPS and LPS strongly induced the expression of IL-10 at analyzed time (24-36 h). (FIG. 12B).

Enhancement of T cell proliferation and activation by PGPS-treated MDDC

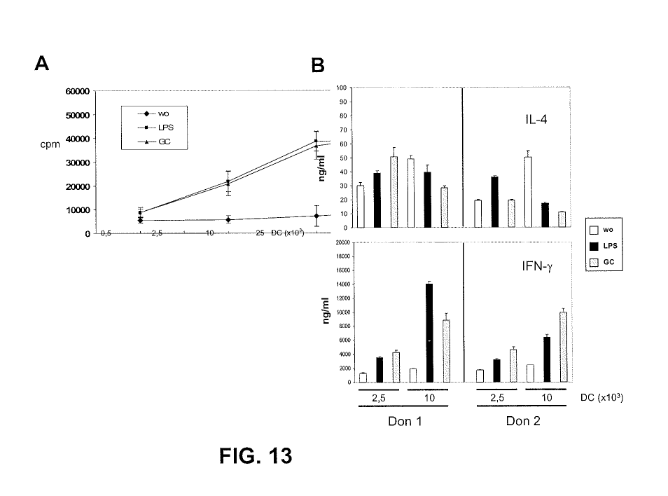

The increased expression of surface markers involved in the presentation of antigen to T cells and increased IL-12 production observed in PGPS-treated MDDC suggested that this compound could induce activation of T cells in allogenic T cell responses. Mature DC has the capacity to induce proliferation in allogenic T cells with a higher efficiency than immature DC^{3,4}. To test this possibility, MDDC were treated with PGPS, washed thoroughly after 24 h, and variable numbers of PGPS-treated DC were incubated for 5 d with a constant number of purified allogeneic T cells. As shown in FIG. 4A, when MDDC were activated with PGPS, they enhanced the proliferation of allogenic T cells at similar levels as LPS-treated MDDC (FIG. 13A). In addition, PGPS-treated MDDC enhanced T cell activation, as evidenced by the increased secretion of IFN-γ into the culture supernatants (FIG. 13B). In contrast, minimal or no changes in IL-4 production were observed upon PGPS or LPS treatments (FIG. 13B).

PGPS up-regulates chemokines and chemokine receptors mRNA expression

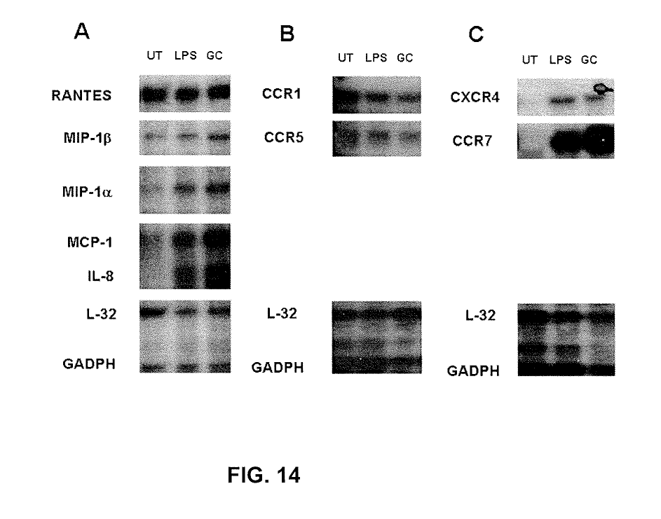

Upon stimulation, DC produce cytokines and chemokines that are involved in leukocyte recruitment^{59,78}. We tested the effect of PGPS on chemokine mRNA production by DC. We observed that PGPS induces an increase in expression of the chemokines MIP-1α, MIP-1 [symbol] IL-8 and MCP-1 mRNA (FIG. 14A). In contrast, PGPS did not modify expression of RANTES and IP-10 mRNA levels (FIG. 14A, and data not shown). Additionally, PGPS also induced the expression mRNA of the chemokine receptors CXCR4 and CCR7 (FIG. 14B), and reduced CCR1 mRNA without affecting CCR5 mRNA levels (FIG. 14C).

PGPS Induces NF-kB Activation, IκB- [symbol] degradation, and MAPK phosphorylation.

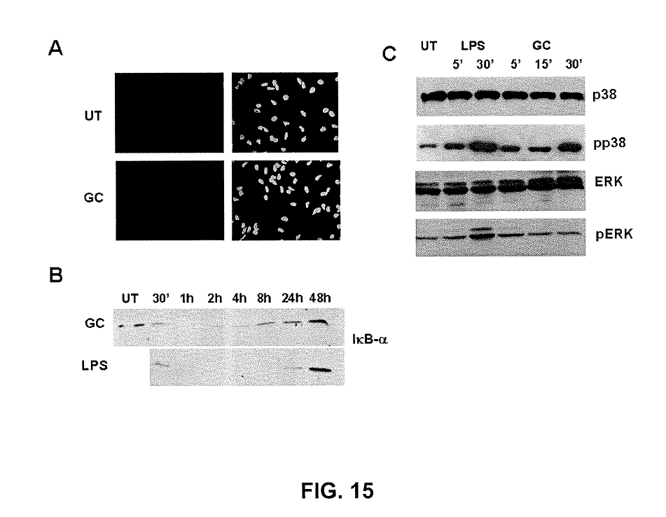

NF-κB activation is a critical step for DC maturation^{7,79}. To determine the molecular mechanism behind the CG-induced MDDC maturation we monitored its ability to trigger NF-κB translocation into the nucleus. Treatment of MDDC with PGPS induced NF-κB (p65/RelA) nuclear translocation, as determined by immunofluorescence experiments (FIG. 15A). Similar results were obtained after treatment of DC with LPS (data not shown).

To further characterize the mechanism of action of PGPS, MDDC were treated with PGPS (1 μg/ml) and LPS (0.1 μg/ml) at different time points and IκB- [symbol] levels were determined by Western blotting. PGPS treatment induced a rapid and transient reduction of IκB-α level, which began to recover after 2 h (FIG. 15B). In contrast, LPS-induced downregulation of IκB-α was not reverted until 24 h post-treatment, which suggested that PGPS exerts a less persistent effect than LPS. Altogether these observations suggest that PGPS-induced maturation is also mediated via NF-κB activation. Additionally, we analyzed MAPK activation in MDDC stimulated with PGPS or LPS by Western blot. As shown in FIG. 6C, PGPS induced the phosphorylation of p38 MAPK in a time-dependent manner and to a similar extent as LPS. Whereas LPS induced a strong phosphorylation of ERK, PGPS treatment induced only a mild ERK phosphorylation. These observations suggest that similar, but not fully identical, activation pathways are promoted by LPS and PGPS on MDDC.

### PGPS Promotes TLR-4-Mediated NF-kB Activation.

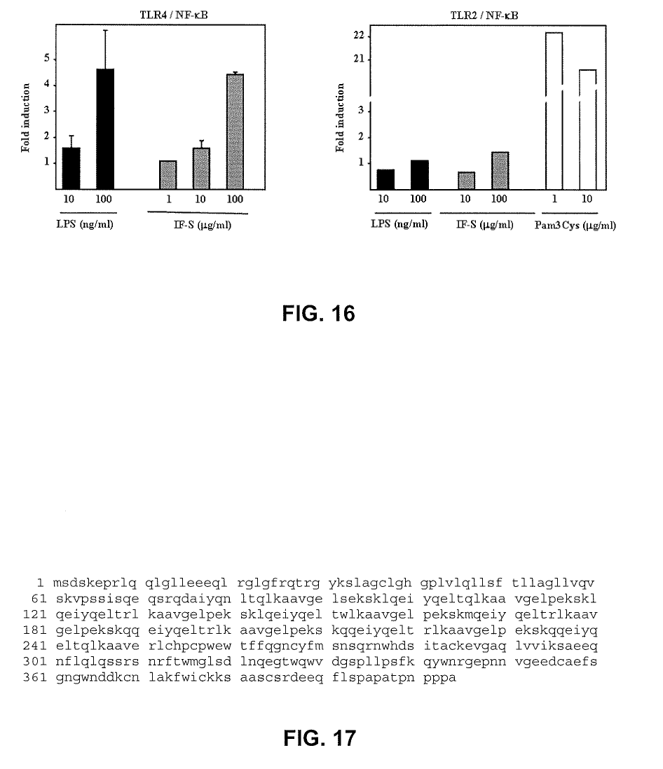

To determine whether TLRs may play a role in the response of MDDC to PGPS, we examine the effect of PGPS in NF-κB activation in HEK293 cells stably transfected with TLR2 or TLR4. These cells were transfected with the NF-κB-Luc reporter construct, stimulated with different PGPS concentrations and assayed for luciferase activity. Additionally, cultures were stimulated with either purified TLR ligands (Pam3Cys for TLR2 or LPS for TLR4). The results showed that TLR4, but nor TLR2 expressing cells, were capable of activating NF-κB in response to PGPS. The transfected TLR2 cells were indeed functional as demonstrated by the ability of the cells to activate NF-κB in response Pam3Cys (FIG. 16).

### EXAMPLE 3

### Search of the Protein and Nucleic Acid Databases for Homologous and Identical or Nearly Identical Protein and Nucleic Acid Sequences

### Materials and Methods

### Software and Databases

Database searching was performed by Internet access using the search algorithms and databases supplied by the National Center for Biotechnology Information (NCBI) (www.ncbi.nlm.nih.gov). The search of the protein and translated nucleic acid database (version: May 7^{th}, 2006) using the Basic Local Alignment Search Tool (BLAST)^{80,81,82}. The search was performed using the protein sequence to human DC-SIGN (FIG. 17) to determine the existence of identical, nearly identical, highly homologous, homologous, similar, or highly similar proteins or translated nucleic acids.

### Results

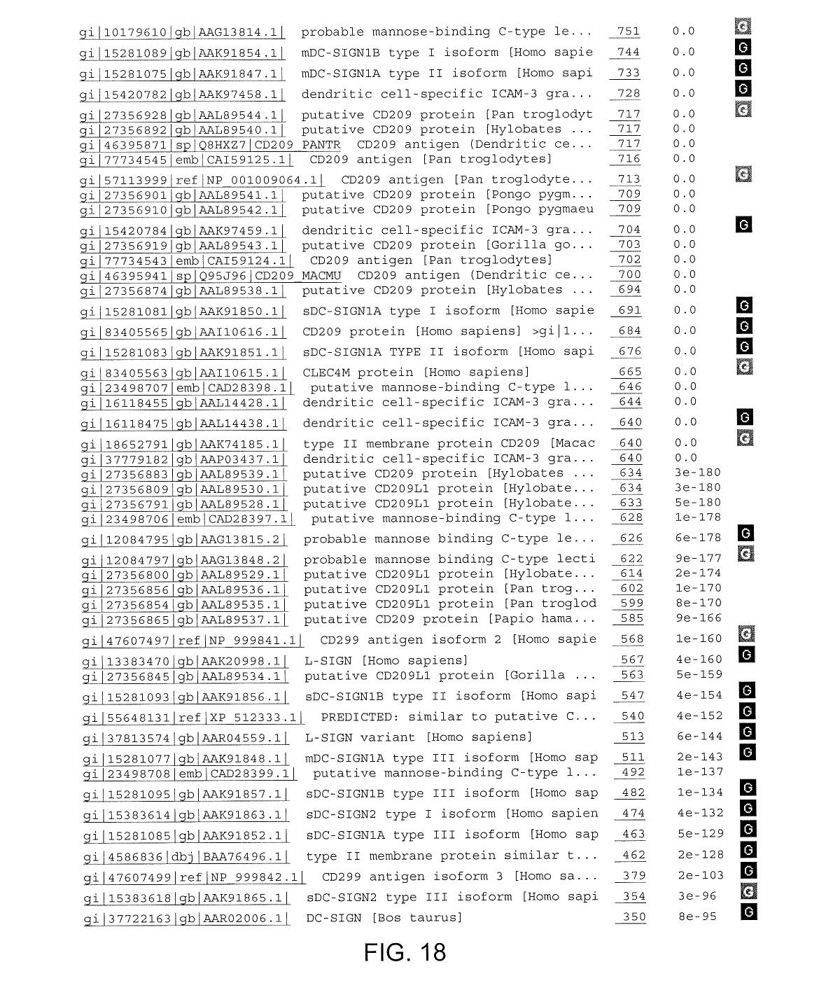

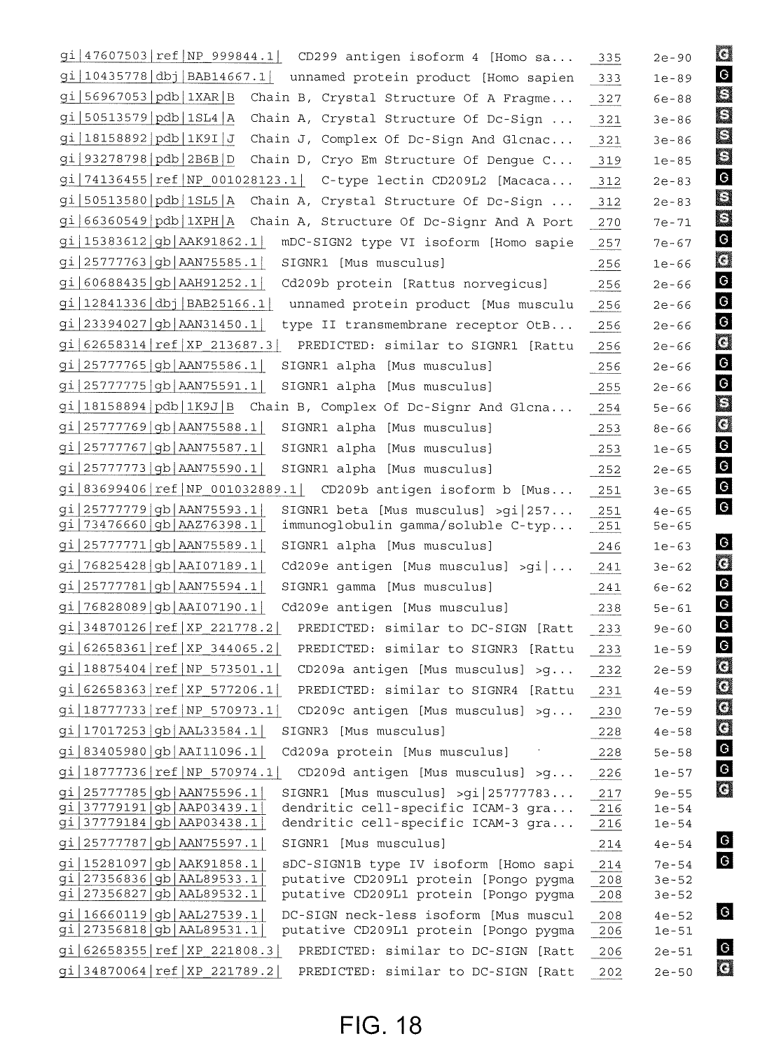

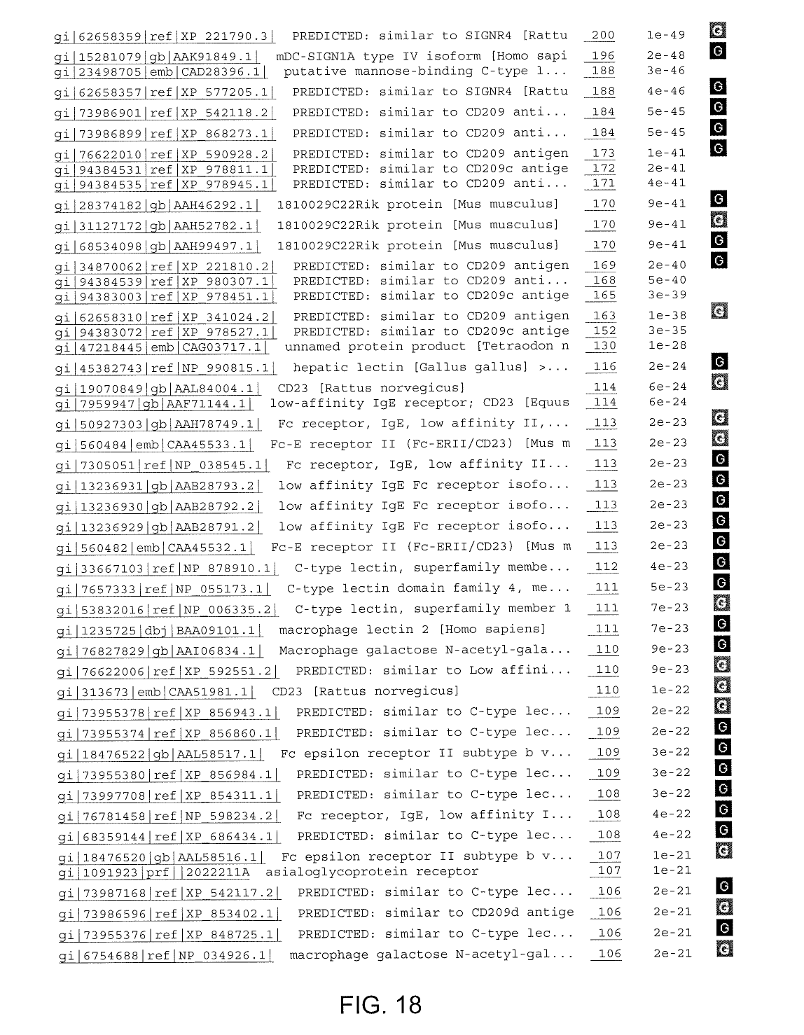

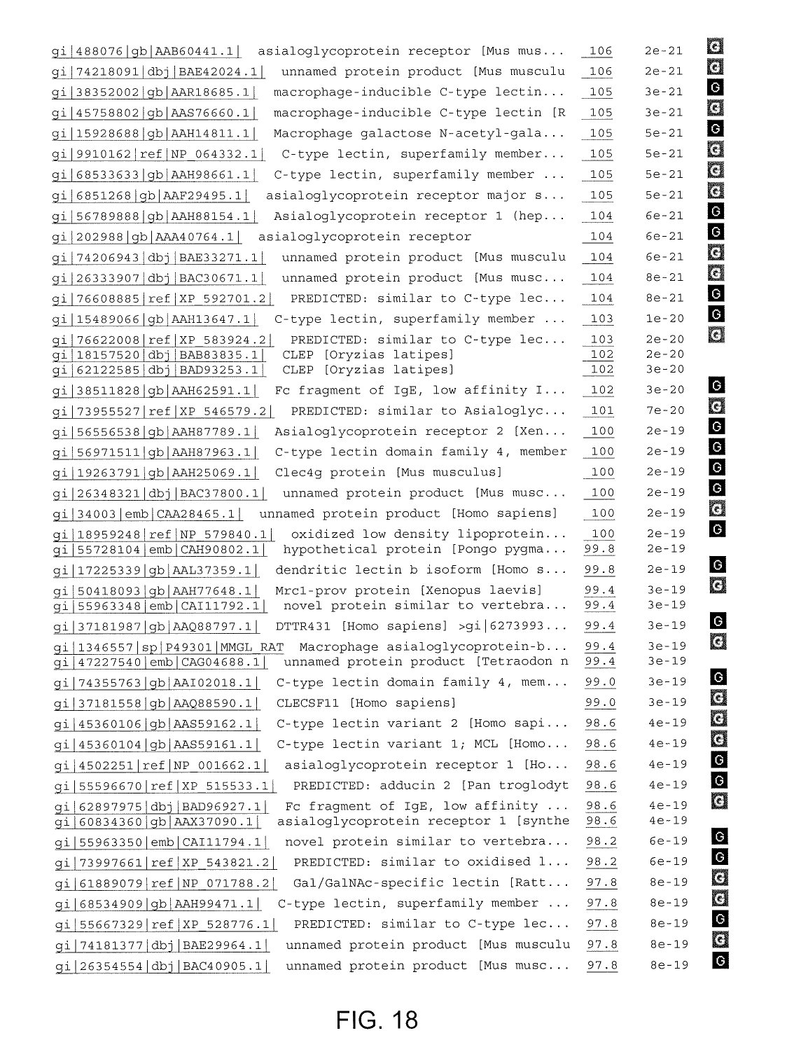

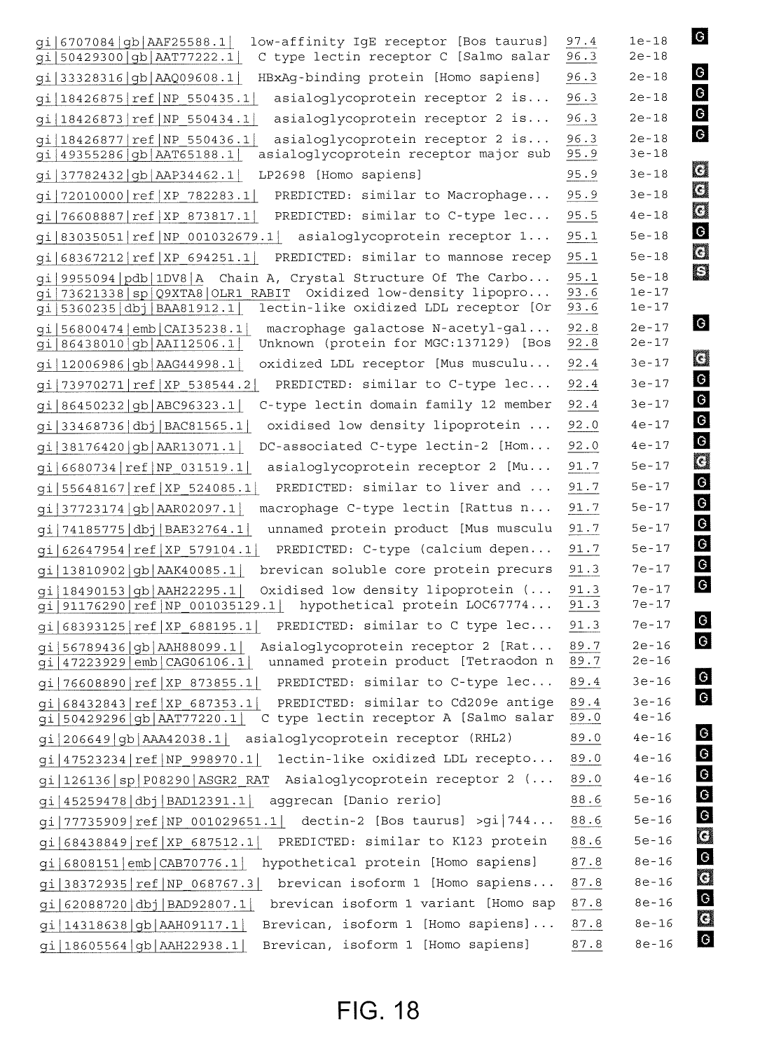

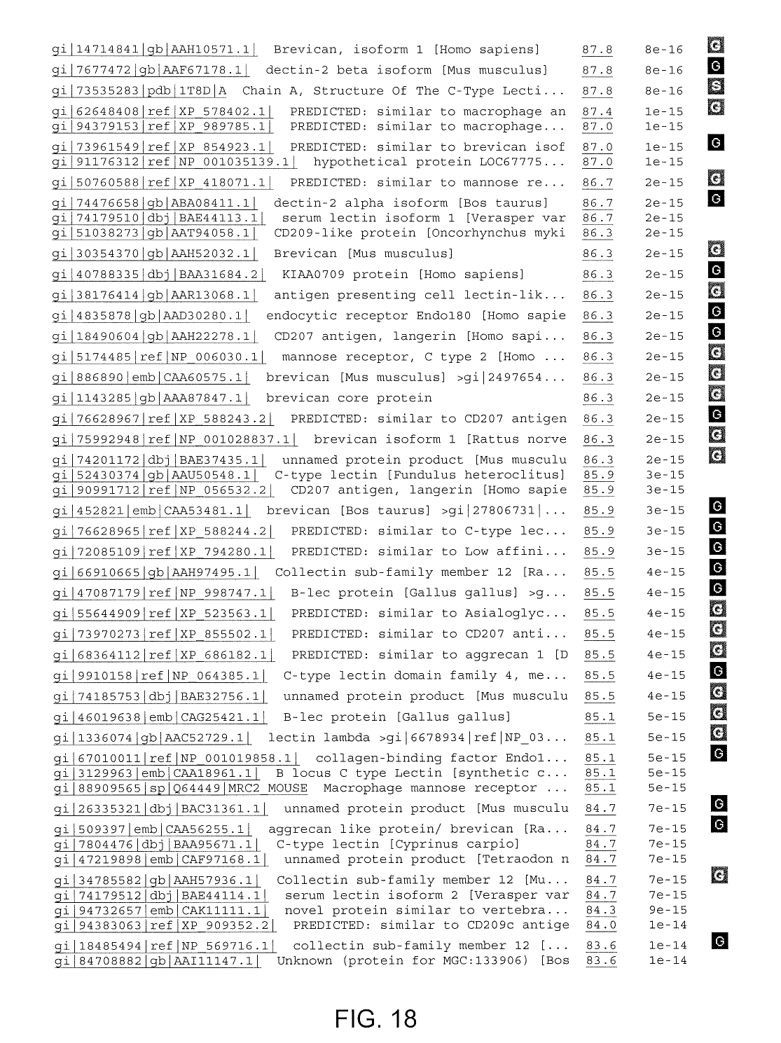

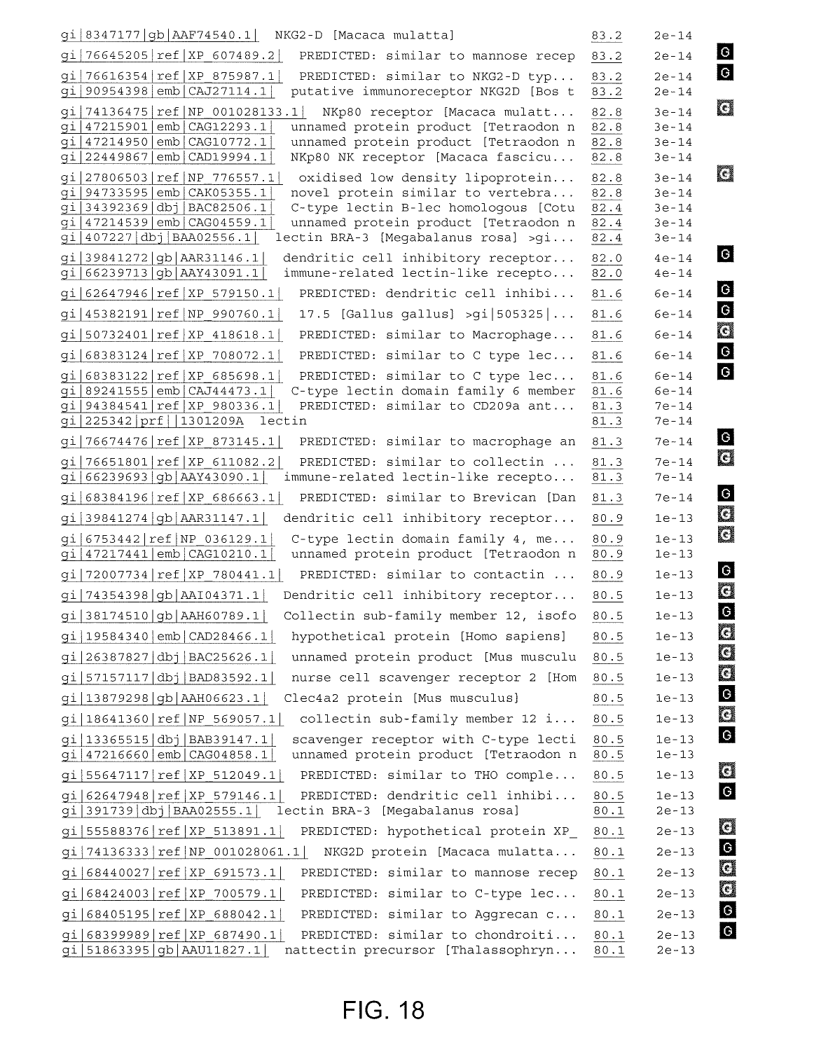

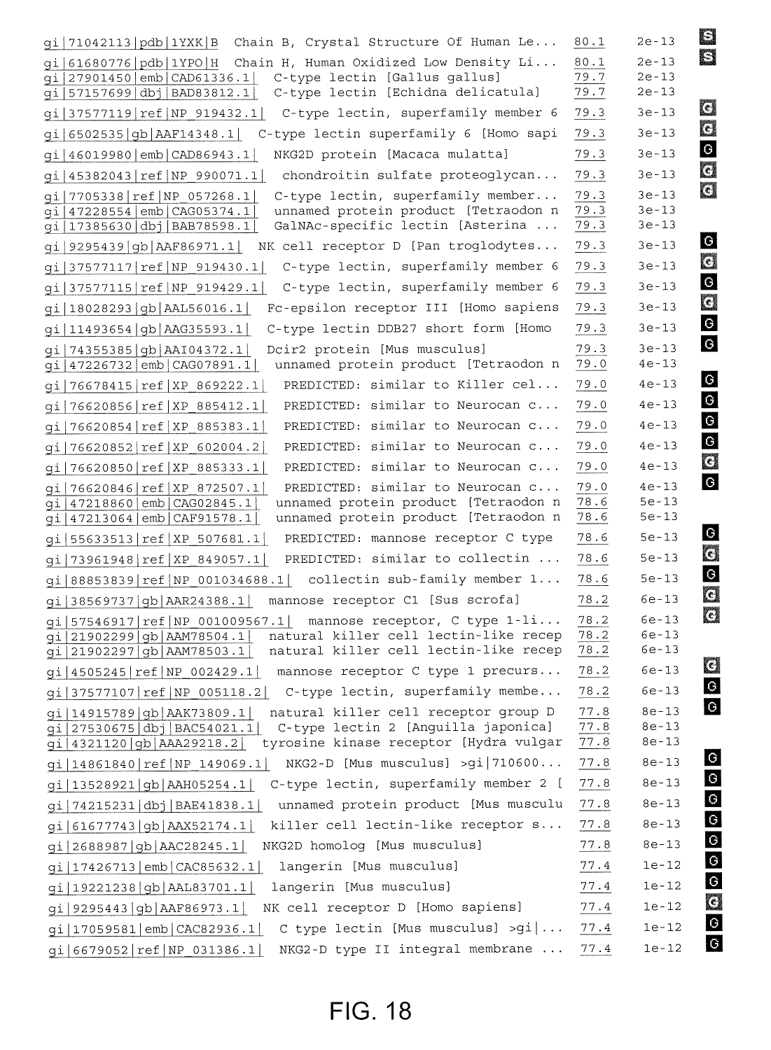

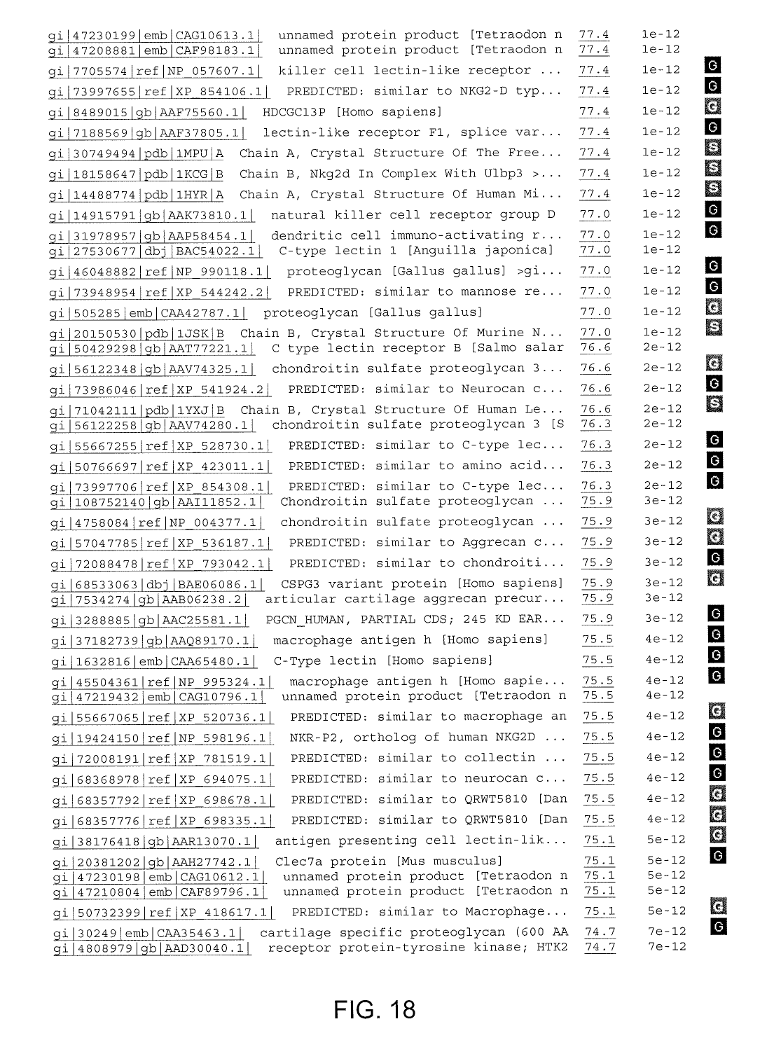

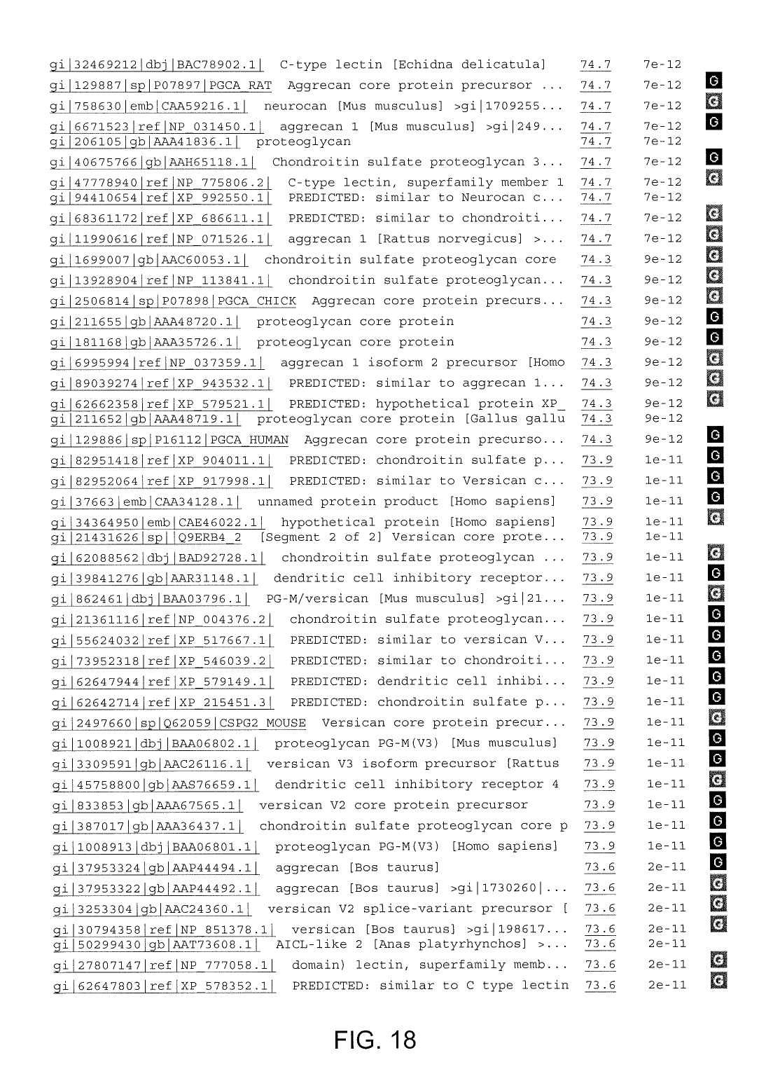

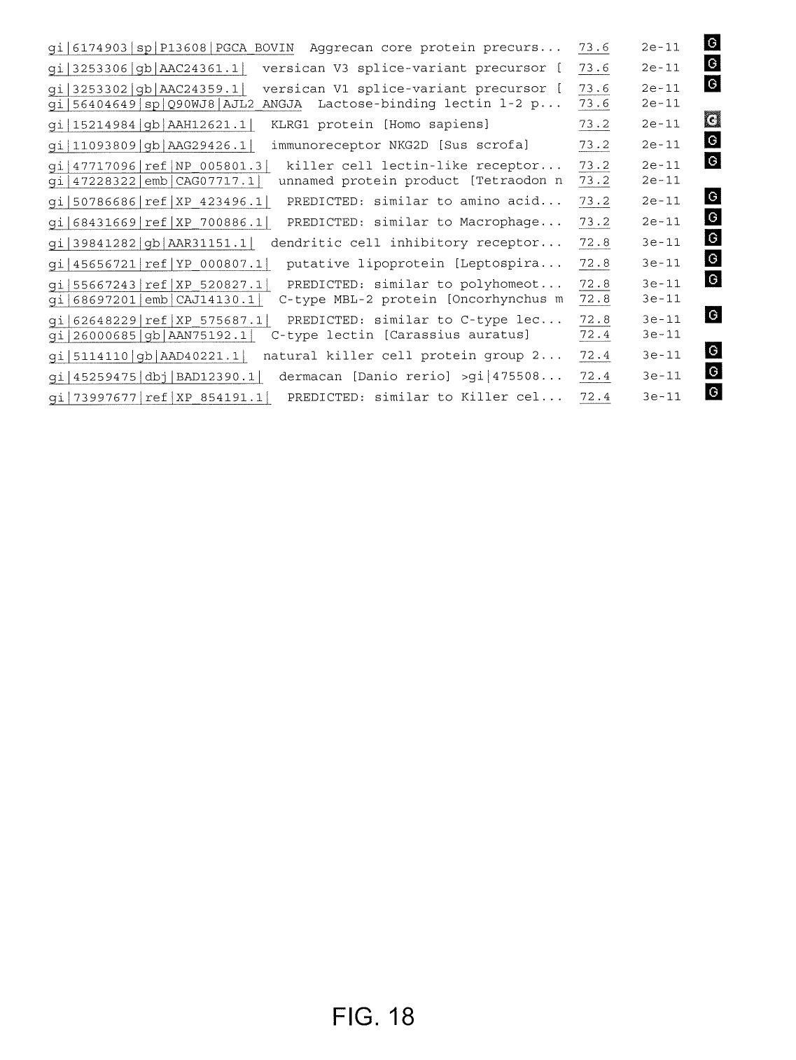

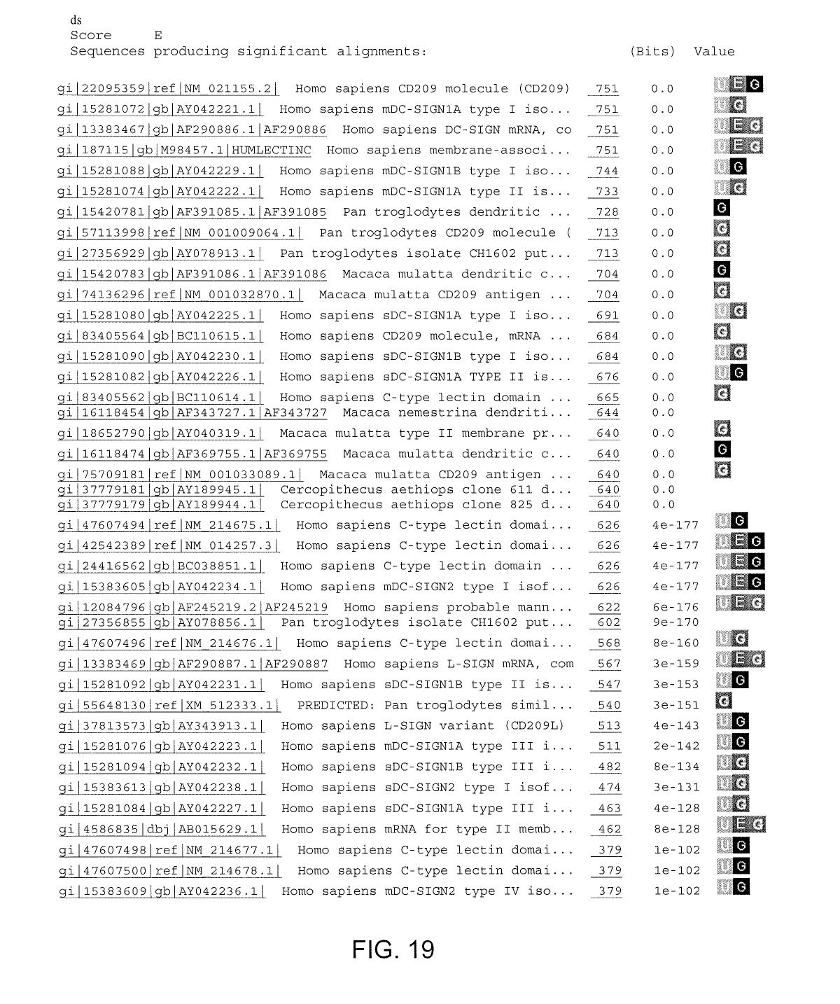

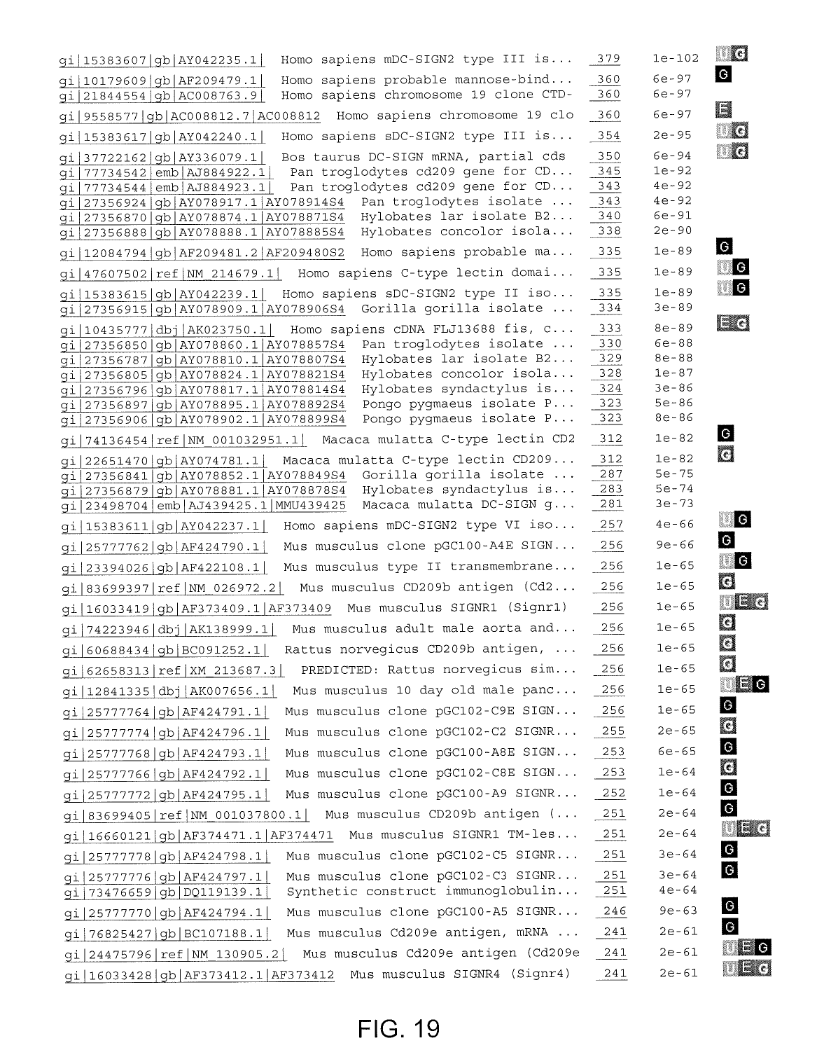

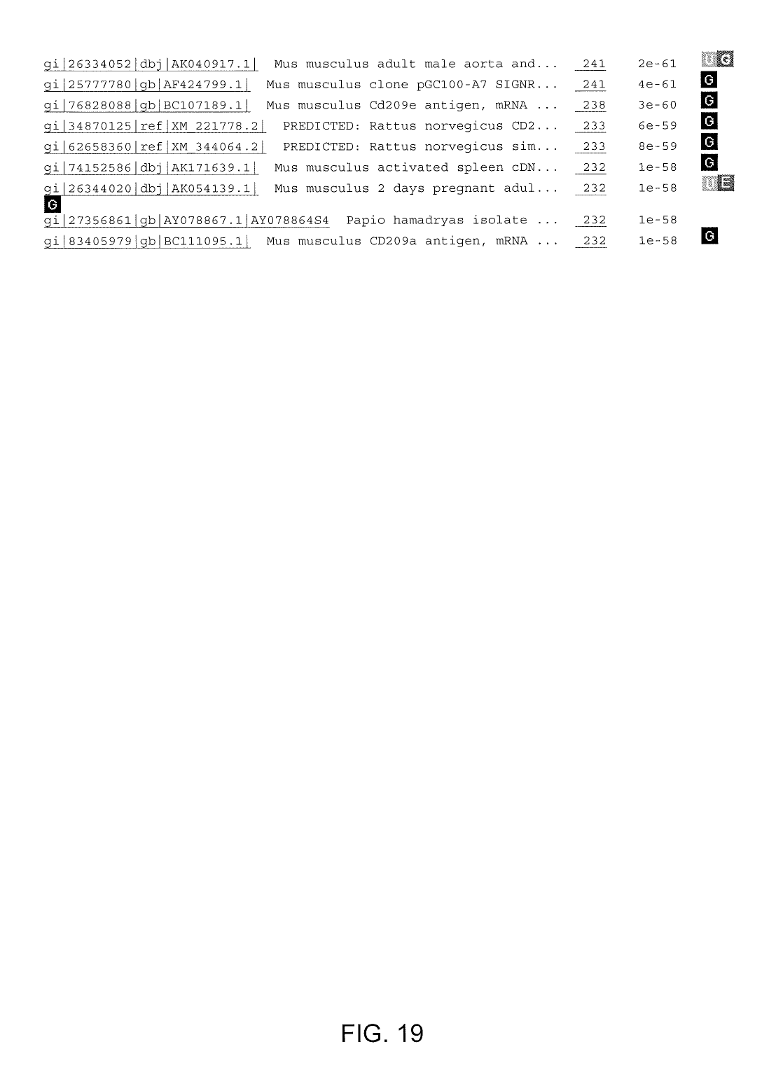

The Basic Local Alignment Search Tool (BLAST) was used to compare the protein sequence for Human DC-SIGN (FIG. 17) against the non-redundant NCBI database using the protein-protein comparison (blastp) function as well as the protein-translated nucleic acid function (tblastn) of the algorithms. The search returned many matches and partial matches (FIG. 18). These sequences each represent DC-SIGN analogues that are likewise implicated in DC-SIGN binding with AM3.

### EXAMPLE 4

### Compositions with PGPS Added to Provide Nutritional Support For Healthy Immune Function

In one aspect, the foregoing results and discussion show that PGPS glycoconjugate materials, such as AM3, benefit the immune system primarily through the PGPS component. Thus, in the case of AM3 and contrary to conventional wisdom, it is not necessary to also include any materials from Ricinus communis, such as the conventional use of storage proteins from nongerminated seeds. It is therefore now advantageously possible to blend the PGPS component of AM3 and/or other PGPS materials, in order to provide functional foods, cosmetics, lotions, and other products that provide nutritional support to benefit healthy immune function.

These materials are generally supplemented with effective amounts of the PGPS materials to benefit the immune function in a range from 0.1 mg to 1 mg per kg of body weight in a target animal, where also more PGPS may be used. Thus, for example, a human animal who is expected to consume a sports drink, food bar, or other functional food that has been supplemented with PGPS might be assessed a dosage on the basis of a range of estimated total body weight, such as from 45 kg to 120 kg. A person at 120 kg would receive an effective amount of PGPS to benefit the immune function, such as 0.1 or 0.2 mg per kg of body weight. These amounts may generally be blended into known functional foods with no adverse effects to the organoleptic qualities of such foods, or they may be used to replace a portion of the carrier, starch, or sugar in such foods.

Delivery forms may also be mixed on the basis of total caloric intake where one rule of thumb is that average people require about fifteen calories per pound of body weight (33 calories per kg). Thus, the PGPS may be mixed with a functional food at a ratio ranging from about 0.1 mg to 1 mg for each 33 calories in the functional food.

“Functional foods” are foods that have been supplemented with materials which may have a functional benefit, such as materials that support healthy immune function, brain function, liver function, digestive function, kidney function, etc. As shown above, PGPS supports healthy immune function. Examples of functional foods that may be prepared to include effective amounts of PGPS include also the food ingredients of protein, starch, sugar, carbohydrate, fats, oils, thickeners, spices salts, and the like that may be individual supplemented with PGPS. By way of example, these food ingredients may be combined as flour mixtures or prepackaged recipes of bread mixes, soup mixes, desert mixes, drink mixes, powdered dietary supplements, and salad dressing mixes, that require final processing by consumers to make a final food product.

Other foods and/or food ingredients that may be prepared to contain PGPS include, by way of example special mixtures of oils and fats as described in U.S. Pat. No. 7,008,661 to Koike for; materials for nutritional compositions for weight management described in U.S. Pat. No. 7,001,618 to Sunvold et al. such as vitamins, amino acids, grain flours of sorghum, barley and/or corn; materials for nutritional compositions for weight management described in U.S. Pat. No. 6,982,098 to Wenniger such as vitamins, herbal supplements, and candy bases made of syrup and sugar; ingredients for the animal feeds described in U.S. Pat. No. 7,070,953 to Bjarnason, et al.; the ingredients disclosed in United States patent Publication 20070148299 to Kihara et al. including such cereal grains as wheat, barley, oats and rye or food or food products with usual raw material of grains in the nature of wheat germ or bran; ingredients for the nutritional dietary system of stern as disclosed in U.S. Pat. No. 6,866,873; any ingredient that may be used in the delivery system for functional ingredients described in U.S. Pat. No. 7,067,150 to Franklin et al. including fats, dietary fiber, protein, carbohydrates, salts, vitamins, minerals and solid materials; and ingredients that may be used in the system for manufacturing dosage forms according to U.S. Pat. No. 6,982,094 to Sowden. The patents of this paragraph are all hereby incorporated by reference to the same extent as though fully replicated herein.

Functional foods may take any form, such as ice cream or other frozen confection as described in: U.S. Pat. No. 7,057,727 to Franklin et al.; milk chocolate mixtures used for dessert coatings as described in U.S. Pat. No. 7,186,435 to Beckett et al.; chocolate for the delivery of functional ingredients as described in U.S. Pat. No. 7,048,941; cereal bars as described in U.S. Pat. No. 7,097,870 to Funk et al., beverages for the delivery of physiologically active substances, like those described in U.S. Pat. Nos. 7,048,959 to Kolisch and 6,866,873 to Portman; pediatric formulae as described in U.S. Pat. No. 6,589,576 to Borschel et al.; instant tea or coffee, and powdered beverage mixes as described in U.S. Pat. No. 4,497,835 to Winston. The patents of this paragraph are all hereby incorporated by reference to the same extent as though fully replicated herein.

In still other examples, cosmetics and topical lotions or solutions may be supplemented with PGPS in roughly the same concentration range as described above to provide nutritional support for healthy skin and provide localized enhancement of the immune function. Examples of such products that may be topically applied include eye drops, ear drops, suntan lotion, lipstick, eyeliner, antibacterial ointments or liquids, cosmetic makeup, deodorant, burn cream, hemorrhoid ointment, analgesic ointment or solution, cocoa butter, face cream, soaps, cleansers, skin care preparations, gels, athlete's foot creams or powders, fingernail polish, moisturizers, shampoo, hair conditioner, perfume, veterinary ointments, wax or chemicals preparations for the removal of hair, medicaments, and insect repellent. These products are generally known to the art in forms without PGPS, and may be prepared by adding the recommended amounts of PGPS to the commercial formulation. Suitable topical materials for mixing with PGPS include, by way of example, the gel sheet cosmetics described in U.S. Pat. No. 7,037,514 to Horizumi and the skin preparations described in U.S. Pat. No. 7,081,254 to Hiraki et al. It will be appreciated that the ingredients used to make these topical materials may be supplemented with PGPS for use in these products, such as a supplemented aloe gel, lanolin, collagen, or a carrier gel for these materials. The patents of this paragraph are all hereby incorporated by reference to the same extent as though fully replicated herein.

The supplementation of cosmetics extends also to the supplementation of ingredients that are mixed to form the cosmetics, such as pigments, emollients, thickeners, preservatives, vitamins, bacteriocides, fungicides, humectants, gels, pH adjusting agents, collagen (especially in micronized form), aldehydes, herbal supplements, botanical extracts, water, alcohol, petrolatum, surfactant, and fragrance.

Dietary supplements may prepared to contain PGPS in the recommended amounts, as dispensed per body weight. Generally speaking the upper limits of the range form 0.1 to 1 mp per kg of body weight may be reasonably

extended with no ill effects, and is observed merely because after a certain point, such as beyond 0.3 mg per kg of body weight, increase amounts of PGPS produce substantially less immunological benefit The PGPS is mixed with ingredients that are used to make powders or pills which are consumed by people

The foregoing discussion teaches by way of example and not by limitation. Insubstantial changes may be made to the precise disclosure without departing from the true scope and spirit of invention. The inventors hereby state their reliance upon the doctrine of equivalents to protect their full rights in the invention.

### REFERENCES

The following documents are incorporated by reference to the same extent as though fully replicated herein:
¹Banchereau, J. and R. M. Steinman, Dendritic cells and the control of immunity. Nature, 1998. 392(6673): p. 245-52.
²Cella M, Sallusto F, Lanzavecchia A. Origin, maturation and antigen presenting function of dendritic cells. Curr Opin Immunol 1997: 9:10-16.
³Banchereau J, Briere F, Caux C, Davoust J, Lebecque S, Liu Y J, Pulendran B, Palucka K. Immunobiology of dendritic cells. Annu Rev Immunol. 2000. 18: p. 767-811.
⁴Cella M, Sallusto F, Lanzavecchia A. Origin, maturation and antigen presenting function of dendritic cells. Curr Opin Immunol 1997: 9: p. 10-16.
⁵de Saint-V is B, Fugier-Vivier I, Massacrier C, Gaillard C, Vanbervliet B, Ait-Yahia S, Banchereau J, Liu Y J, Lebecque S, Caux C. The cytokine profile expressed by human dendritic cells is dependent on cell subtype and mode of activation. J. Immunol. 1998; 160(4): p. 1666-76.
⁶Moser B, Wolf M, Walz A, Loetscher P. Chemokines: multiple levels of leukocyte migration control. Trends Immunol. 2004; 25(2): p. 75-84.
⁷Ardeshna K M, Pizzey A R, Devereux S, Khwaja A. The PI3 kinase, p38 SAP kinase, and NF-kB signal transduction pathways are involved in the survival and maturation of lipopolysaccharide-stimulated human monocyte-derived dendritic cells. Blood 2000; 96:p. 1039-1047.
⁸Neumann M, Fries H W, Scheicher, Keikavoussi P, Kolb-Maürer A, Bröcker E B, Serfling E, Kämpgen E. Differential expression of Rel/NF-kB and octamer factors is a hallmark of the generation and maturation of dendritic cells. Blood 2000;95: p. 277-285.
⁹Arrighi J F, Rebsamen M, Rousset F, Kindler V, Hauser C. A critical role for p38 mitogen-activated protein kinase in the maturation of human blood-derived dendritic cells induced by lipopolysaccharide, TNF-α, and contact sensitizers. J Immunol 2001;166: p. 3837-3845.
¹⁰Sato K, Nagayama H, Tadokoro K, Juji T, Takahashi T A. Extracellular signal-regulated kinase, stress-activated protein kinase/c-Jun N-terminal kinase, and p38mapk are involved in IL-10-mediated selective repression of TNF-a-induced activation and maturation of human peripheral blood monocyte-derived dendritic cells. J Immunol 1999:162; p. 3865-3872.
¹¹Steinman, R. M., D. Hawiger, and M. C. Nussenzweig, Tolerogenic dendritic cells. Annu Rev Immunol, 2003. 21: p. 685-711.
¹²Takeda, K., T. Kaisho, and S. Akira, Toll-like receptors. Annu Rev Immunol, 2003. 21: p. 335-76.
¹³van Kooyk, Y. and T. B. Geijtenbeek, DC-SIGN: escape mechanism for pathogens. Nat Rev Immunol, 2003. 3(9): p. 697-709.
¹⁴Curtis, B. M., S. Scharnowske, and A. J. Watson, Sequence and expression of a membrane-associated C-type lectin that exhibits CD4-independent binding of human immunodeficiency virus envelope glycoprotein gp120. Proc Natl Acad Sci USA, 1992. 89(17): p. 8356-60.
¹⁵Geijtenbeek, T. B., et al., Identification of DC-SIGN, a novel dendritic cell-specific ICAM-3 receptor that supports primary immune responses. Cell, 2000. 100(5): p. 575-85.
¹⁶Engering, A., et al., Subset of DC-SIGN(+) dendritic cells in human blood transmits HIV-1 to T lymphocytes. Blood, 2002. 100(5): p. 1780-6.
¹⁷Geijtenbeek, T. B., et al., DC-SIGN, a dentritic cell-specific HIV-1 receptor present in placenta that infects T cells in trans-a review. Placenta, 2001. 22 Suppl A: p. S19-23.
¹⁸Alvarez, C. P., et al., C-type lectins DC-SIGN and L-SIGN mediate cellular entry by Ebola virus in cis and in trans. J Virol, 2002. 76(13): p. 6841-4.
¹⁹Pohlmann, S., et al., Hepatitis C virus glycoproteins interact with DC-SIGN and DC-SIGNR. J Virol, 2003. 77(7): p. 4070-80.
²⁰Lozach, P. Y., et al., DC-SIGN and L-SIGN are high affinity binding receptors for hepatitis C virus glycoprotein E2. J Biol Chem, 2003. 278(22): p. 20358-66.
²¹Tassaneetrithep, B., et al., DC-SIGN(CD209) mediates dengue virus infection of human dendritic cells. J Exp Med, 2003. 197(7): p. 823-9.
²²Colmenares, M., et al., Dendritic cell (DC)-specific intercellular adhesion molecule 3 (ICAM-3)-grabbing nonintegrin (DC-SIGN, CD209), a C-type surface lectin in human DCs, is a receptorfor Leishmania amastigotes. J Biol Chem, 2002. 277(39): p. 36766-9.
²³Tailleux, L., et al., DC-SIGN is the major Mycobacterium tuberculosis receptor on human dendritic cells. J Exp Med, 2003. 197(1): p. 121-7.
²⁴Geijtenbeek, T. B., et al., Mycobacteria target DC-SIGN to suppress dendritic cell function. J Exp Med, 2003. 197(1): p. 7-17.
²⁵Cambi, A., et al., The C-type lectin DC-SIGN(CD209) is an antigen-uptake receptorfor Candida albicans on dendritic cells. Eur J Immunol, 2003. 33(2): p. 532-8.
²⁶Serrano-Gomez, D., et al., Dendritic cell-specific intercellular adhesion molecule 3-grabbing nonintegrin mediates binding and internalization of Aspergillus fumigatus conidia by dendritic cells and macrophages. J Immunol, 2004. 173(9): p. 5635-43.
²⁷Feinberg, H., et al., Structural basis for selective recognition of oligosaccharides by DC-SIGN and DC-SIGNR. Science, 2001. 294(5549): p. 2163-6.
²⁸Frison, N., et al., Oligolysine-based oligosaccharide clusters: selective recognition and endocytosis by the mannose receptor and dendritic cell-specific intercellular adhesion molecule 3 (ICAM-3)-grabbing nonintegrin. J Biol Chem, 2003. 278(26): p. 23922-9.
²⁹Geijtenbeek, T. B., et al., DC-SIGN-ICAM-2 interaction mediates dendritic cell trafficking. Nat Immunol, 2000.1(4): p. 353-7.
³⁰van Gisbergen, K. P., et al., Neutrophils mediate immune modulation of dendritic cells through glycosylation-dependent interactions between Mac-1 and DC-SIGN. J Exp Med, 2005. 201(8): p. 1281-92.
³¹Alvarez-Mon, M., et al., Treatment with the immunomodulator AM3 improves the health-related quality of life of patients with COPD. Chest, 2005. 127(4): p. 1212-8.
³²Brieva, A., A. Guerrero, and J. P. Pivel, Inmunoferon, a glycoconijugate of natural origin, regulates the liver response to inflammation and inhibits TNF-alpha production by an HPA axis-dependent mechanism. Int Immunopharmacol, 2002. 2(6): p. 807-13.
³³Varela, J., et al., Identification and characterization of the peptidic component of the immunomodulatory glycoconjugate Immunoferon. Methods Find Exp Clin Pharmacol, 2002. 24(8): p. 471-80.
³⁴Rojo, J. M., et al., Enhancement of lymphocyte proliferation, interleukin-2 production and NK activity by immunoferon (AM-3), a fungal immunomodulator: variations in normal and immunosuppressed mice. Int J Immunopharmacol, 1986. 8(6): p. 593-7.
³⁵Sanchez, L., et al., AM3, an adjuvant to hepatitis B revaccination in non-responder healthy persons. J Hepatol, 1995. 22(1): p. 119-21.
³⁶Perez-Garcia, R., et al., AM3 (Immunoferon) as an adjuvant to hepatitis B vaccination in hemodialysis patients. Kidney Int, 2002. 61(5): p. 1845-52.
³⁷Prieto, A., et al., Defective natural killer and phagocytic activities in chronic obstructive pulmonary disease are restored by glycophosphopeptical (immunoferon). Am J Respir Crit Care Med, 2001. 163(7): p. 1578-83.
³⁸Brieva, A., et al., Immunoferon, a glycoconjugate of natural origin, inhibits LPS-induced TNF-alpha production and inflammatory responses. Int Immunopharmacol, 2001. 1(11): p. 1979-87.
³⁹Majano, P., et al., AM3 inhibits LPS-induced iNOS expression in mice. Int Immunopharmacol, 2005. 5(7-8): p. 1165-70.
⁴⁰Majano et al., manuscript submitted, 2006
⁴¹Majano et al, unpublished observations
⁴²Brieva et al, unpublished observations
⁴³Majano P, Roda-Navarro P, Alonso-Lebrero J L, Brieva A, Casal C, Pivel J P, Lopez-Cabrera M, Moreno-Otero R. AM3 inhibits HBV replication through activation of peripheral blood mononuclear cells. Int Immunopharmacol 2004;4(7): p. 921-927.
⁴⁴Ramamoorthy, L., M. C. Kemp, and I. R. Tizard, Acemannan, a beta-(1,4)-acetylated mannan, induces nitric oxide production in macrophage cell line RAW 264.7. Mol Pharmacol, 1996. 50(4): p. 878-84.
⁴⁵Nigou, J., et al., Mycobacterial lipoarabinomannans: modulators of dendritic cell function and the apoptotic response. Microbes Infect, 2002. 4(9): p. 945-53.
⁴⁶Meyer, S., et al., DC-SIGN mediates binding of dendritic cells to authentic pseudo-Lewis Y glycolipids of Schistosoma mansoni cercariae, the first parasite-specific ligand of DC-SIGN. J Biol Chem, 2005. 280(45): p. 37349-59.
⁴⁷van Kooyk, Y., et al., Pathogens use carbohydrates to escape immunity induced by dendritic cells. Curr Opin Immunol, 2004. 16(4): p. 488-93.
⁴⁸Engering, A., et al., The dendritic cell-specific adhesion receptor DC-SIGN internalizes antigen for presentation to T cells. J Immunol, 2002. 168(5): p. 2118-26.
⁴⁹Tacken, P. J., et al., Effective induction of naive and recall T-cell responses by targeting antigen to human dendritic cells via a humanized anti-DC-SIGN antibody. Blood, 2005. 106(4): p. 1278-85.
⁵⁰Schjetne, K. W., et al., A mouse C kappa-specific T cell clone indicates that DC-SIGN is an efficient target for antibody-mediated delivery of T cell epitopes for MHC class II presentation. Int Immunol, 2002. 14(12): p. 1423-30.
⁵¹Meyer-Wentrup, F., et al., “Sweet talk”: closing in on C type lectin signaling. Immunity, 2005. 22(4): p. 399-400.
⁵²Rogers, N. C., et al., Syk-dependent cytokine induction by Dectin-1 reveals a novel pattern recognition pathway for C type lectins. Immunity, 2005. 22(4): p. 507-17.
⁵³Caparros, E., et al., DC-SIGN ligation on dendritic cells results in ERK and PI3K activation and modulates cytokine production. Blood, 2006. in press.
⁵⁴Kalinski P, Hilkens C M, Wierenga E A, Kapsenberg M L. T-cell priming by type-1 and type-2 polarixed dendritic cells: the concept of a third signal. Immunol Today. 1999;20: 561-567.
⁵⁵Gately M K, Renzetti L M, Magram J, Stem A S, Adorini L, Gubler U, Presky D H. The interleukin-12/interleukin-12-receptor system: role in normal and pathologic immune responses. Annu Rev Immunol 1998;16:p. 495-521.
⁵⁶Moore K W, de Waal Malefyt R, Coffman R, O'Garra A. Interleukin-10 and interleukin-10 receptor. Annu Rev Immunol 2001; 19: p. 683-765.
⁵⁷Langenkamp A, Messi M, Lanzavecchia A, Sallusto F. Kinetics of dendritic cell activation: impact on priming of TH1, TH2 and nonpolarized T cells. Nat Immunol 2000;1:p. 311-316.
⁵⁸Corinti S, Albanesi C, la Sala A, Pastore S, Girolomoni G. Regulatory activity of autocrine IL-10 on dendritic cell functions. J Immunol 2001;166:p. 4312-4318.
⁵⁹Sallusto F, Schaerli P, Loetscher P, Schaniel C, Lenig D, Mackay C R, Qin S and Lanzavecchia A. Rapid and coordinated switch in chemokine receptor expression during dendritic cell maturation. Eur J Immunol 1998. 28: p. 2760-2769.
⁶⁰Re F, Strominger J L. Toll-like receptor 2 (TLR2) and TLR4 differentially activate human dendritic cells. J Biol Chem. 2001; 276(40):p. 37692-37699.
⁶¹Agrawal S, Agrawal A, Doughty B, Gerwitz A, Blenis J, Van Dyke T, Pulendran B. Different Toll-like receptor agonists instruct dendritic cells to induce distinct Th responses via differential modulation of extracellular signal-regulated kinase-mitogen-activated protein kinase and c-Fos. J. Immunol. 2003 Nov. 15; 171(10):p. 4984-4989.
⁶²An H, Yu Y, Zhang M, Xu H, Qi R, Yan X, Liu S, Wang W, Guo Z, Guo J, Qin Z, Cao X. Involvement of ERK, p38 and NF-kappaB signal transduction in regulation of TLR2, TLR4 and TLR9 gene expression induced by lipopolysaccharide in mouse dendritic cells. Immunology 2002; 106(1):p. 38-45.
⁶³Rescigno et al, 2001
⁶⁴Karima R, Matsumoto S, Higashi H, Matsushima K. The molecular pathogenesis of endotoxic shock and organ failure. Mol Med Today 1999; 5:123-32.
⁶⁵Brieva et al, 2004
⁶⁶Colmenares, M., et al., The dendritic cell receptor DC-SIGN discriminates among species and life cycle forms of Leishmania. J Immunol, 2004. 172(2): p. 1186-90.
⁶⁷Geijtenbeek, T. B., et al., DC-SIGN, a dendritic cell-specific HIV-1-binding protein that enhances trans-infection of T cells. Cell, 2000. 100(5): p. 587-97.
⁶⁸Yoshitomi, H., et al., A role for fungal {beta}-glucans and their receptor Dectin-1 in the induction of autoimmune arthritis in genetically susceptible mice. J Exp Med, 2005. 201(6): p. 949-60
⁶⁹Geijtenbeek, T. B., et al., DC-SIGN-ICAM-2 interaction mediates dendritic cell trafficking. Nat Immunol, 2000.1(4): p. 353-7.
⁷⁰van Gisbergen, K. P., et al., Neutrophils mediate immune modulation of dendritic cells through glycosylation-dependent interactions between Mac-1 and DC-SIGN. J Exp Med, 2005. 201(8): p. 1281-92.
⁷¹de la Rosa, G., et al., Regulated recruitment of DC-SIGN to cell-cell contact regions during zymosan-induced human dendritic cell aggregation. J Leukoc Biol, 2005. 77(5): p. 699-709.
⁷²Claasen, B., et al., Direct observation of ligand binding to membrane proteins in living cells by a saturation transfer double difference (STDD) NMR spectroscopy method shows a significantly higher affinity of integrin alpha(IIb)beta3 in native platelets than in liposomes. J Am Chem Soc, 2005. 127(3): p. 916-9.
⁷³Mayer, M. and B. Meyer, Group epitope mapping by saturation transfer difference NMR to identify segments of a ligand in direct contact with a protein receptor. J Am Chem Soc, 2001. 123(25): p. 6108-17.
⁷⁴Mari, S., et al., 1D saturation transfer difference NMR experiments on living cells: the DC-SIGN/oligomannose interaction. Angew Chem Int Ed Engl, 2004. 44(2): p. 296-8.
⁷⁵Puig-Kröger A, Relloso M, Femandez-Capetillo O, Zubiaga A, Silva A, Bemabeu C, Corbi A. Extracellular signal-regulated protein kinase signaling pathway negatively regulates the phenotypic and functional maturation of monocyte-derived human dendritic cells. Blood 2001;98: p. 2175-2182.
⁷⁶Yano O, Kanellopoulos J, Kieran M, Le Bail O, Israel A, Kourilsky P. Purification of KBF1, a common factor binding to both H-2 and beta 2-microglobulin enhancers. EMBO J. 1987; 6(11): p. 3317-3324.
⁷⁷Sallusto F, Lanzavecchia A. Efficient presentation of soluble antigen by cultured human dendritic cells is maintained by granulocyte/macrophage colonystimulating factor plus interleukin 4 and down-regulated by tumor necrosis factor alpha. J Exp Med 1994;179: p. 1109-1118.
⁷⁸Sallusto F, Palermo B, Lenig D, Miettinen M, Matikainen S, Julkunen I, Forster R, Burgstahler R, Lipp M, Lanzavecchia A. Distinct patterns and kinetics of chemokine production regulate dendritic cell function. Eur J Immunol. 1999; 29(5): p. 1617-25
⁷⁹Neumann M, Fries H W, Scheicher, Keikavoussi P, Kolb-Maürer A, Brocker E B, Serfling E, Kämpgen E. Differential expression of Rel/NF-kB and octamer factors is a hallmark of the generation and maturation of dendritic cells. Blood 2000; 95: p. 277-285.
⁸⁰Madden, Tom. The BLAST Sequence Analysis Tool. http://www.ncbi.nlm.nih.gov/Education/BLASTinfo/information3.html
⁸¹Altschul S F, Gish W, Myers E W, Lipman D J. Basic Local Alignment Search Tool. J Mol Biol 1990; 215: p. 403-410.
⁸²Altschul S F, Madden T L, Schaffer A A, Zhang Z, Miller W, Lipman D J. Gapped BLAST and PSI-BLAST a new generation of protein database search programs. Nucleic Acid Res; 1997: p. 3389-3402.

Claimed are:

1. A composition for enhancing immune function, comprising:
a mannan polysaccharide complex carbohydrate having a capacity to bind with DC-SIGN,
the mannan polysaccharide complex carbohydrate being present in an effective amount for immunomodulation of the immune system; and
a co-active agent for stimulating an immune response,
the co-active agent being combined with the mannan polysaccharide complex for increased benefit of the immune response by immunomodulation from the mannan polysaccharide complex.

2. The composition of claim 1 wherein the mannan polysaccharide complex carbohydrate is a phosphorylated glucomannan polysaccharide.

3. The composition of claim 2 wherein the mannan polysaccharide complex carbohydrate is derived from Candida utilis.

4. The composition of claim 1 wherein the mannan polysaccharide complex carbohydrate is derived from a fungus.

5. The composition of claim 1 wherein the mannan polysaccharide complex carbohydrate is derived from a plant.

6. The composition of claim 1 wherein the co-active agent includes a vaccine.

7. The composition of claim 6 wherein the vaccine is formulated to provide immunity against a pathogen that binds with DC-SIGN.

8. The composition of claim 7 wherein the pathogen includes at least one member is selected from the group consisting of HIV-1, Ebola virus, Leishmania pifanoi, Cytomegalovirus, Hepatitis C, Dengue virus, Helicobacter pylori, Klebsiella pneumonae, Mycobacterium, Mycobacterium tuberculosis, Schistosoma mansoni, and Coxiella burnetii.

9. The composition of claim 1 wherein the co-active agent includes a treating agent for infectious disease.

10. The composition of claim 1 wherein the treating agent for infectious disease includes an antibiotic.

11. The composition of claim 1 wherein the antibiotic. includes at least one member selected from the group consisting of aminoglycosides including amikacin, gentamicin, kanamycin, neomycin, netilmicin, streptomycin, and tobramycin; carbacephems including loracarbef, ertapenem, imipenem/cilastatin, and meropenem; cephalosporins including cefadroxil, cefazolin, cephalexin; cefaclor, cefamandole, cefoxitin, cefprozil, cefuroxime, cefixime, cefdinir, cefditoren, cefoperazone, cefotaxime, claforan, cefpodoxime, ceftazidime ceftibuten, ceftizoxime, ceftriaxone, cefepime, and maxipime; glycopeptides including teicoplanin and vancomycin; macrolides including azithromycin, clarithromycin, dirithromycin, eythromycin, and troleandomycin; monobactam inclosing aztreonam; penicillins inclosing amoxicillin, ampicillin, azlocillin, carbenicillin, cloxacillin, dicloxacillin, flucloxacillin, mezlocillin, nafcillin, penicillin, piperacillin, and ticarcillin; polypeptides including bacitracin, colistin, and polymyxin B; quinolones including ciprofloxacin, enoxacin, gatifloxacin, levofloxacin, lomefloxacin, moxifloxacin, norfloxacin, ofloxacin, and trovafloxacin; sulfonamides incluing mafenide, prontosil, sulfacetamide, sulfamethizole, sulfanilimide, sulfasalazine, sulfisoxazole, trimethoprim, and trimethoprim-sulfamethoxazole; tetracyclines including demeclocycline, doxycycline, minocycline, oxytetracycline, and tetracycline; and others including chloramphenicol, clindamycin, ethambutol, fosfomycin, furazolidone, isoniazid, linezolid, metronidazole, nitrofurantoin, pyrazinamide, quinupristin/dalfopristin, rifampin and spectinomycin.

12. The composition of claim 1 wherein the co-active agent includes a nutrient that provides support for beneficial immune response.

13. The composition of claim 12, wherein the nutrient includes at least one member selected from the group consisting of vitamins including vitamins A, B-6, biotin, C, D, and E.

14. The composition of claim 12, wherein the nutrient includes at least one member selected from the group consisting of minerals including Cu, Fe, Se, Cr, Co, Zn, and salts thereof.

15. The composition of claim 1 formulated with a pharmacologically compatible materials for oral administration.

16. The composition of claim 1 formulated with a pharmacologically compatible materials for nasal administration.

17. The composition of claim 1 formulated with a pharmacologically compatible materials for injectable administration.

18. The composition of claim 1 formulated with a pharmacologically compatible materials for topical administration.

19. The composition of claim 1 formulated as a food product other than a capsule or tablet.

20. A method for immunomodulation of the immune system comprising:
delivering internally to an animal a composition that contains
a mannan polysaccharide complex carbohydrate having a capacity to bind with DC-SIGN,
the mannan polysaccharide complex carbohydrate being present in an effective amount for immunomodulation of the immune system; and
a co-active agent for stimulating an immune response, the co-active agent being combined with the mannan polysaccharide complex for increased benefit of the immune response by immunomodulation from the mannan polysaccharide complex; and
allowing the composition to work on the animal to produce the immune response and the immunomodulation.

21. The method of claim 20 wherein the mannan polysaccharide complex carbohydrate used in the step of delivering includes a phosphorylated glucomannan polysaccharide.

22. The method of claim 21 wherein the phosphorylated glucomannan polysaccharide is derived from Candida utilis.

23. The method of claim 20, prior to the step of delivering, further comprising a step of diagnosing the animal with an infectious condition that is caused by a pathogen and is in need of treatment.

24. The method of claim 23 wherein the pathogenesis of the pathogen includes binding to DC-SIGN.

25. The method of claim 23 wherein the pathogen is a fungus.

26. The method of claim 23 wherein the pathogen is a parasite.

27. The method of claim 23 wherein the pathogen is a virus.

28. The method in claim 23 wherein the pathogen is a bacterium.

29. The method of claim 23 wherein the pathogen is a prion.

30. The method of claim 23 wherein the pathogen is selected from the species consisting of Candida, Aspergillus, Mycobacterium, Pneumocistis, Schistosoma and Leishmania.

31. The method of claim 23 wherein the pathogen is a virus as Ebola, HIV, or Hepatitis C.

32. The method of claim 23, wherein the pathogen includes at least one member is selected from the group consisting of HIV-1, Ebola virus, Leishmania pifanoi, Cytomegalovirus, Hepatitis C, Dengue virus, Helicobacter pylori, Klebsiella pneumonae, Mycobacterium tuberculosis, Schistosoma mansoni, and Coxiella burnetii.

33. The method of claim 20 wherein the mannan polysaccharide is capable of binding with a pattern recognition molecule inclosing lectins, toll like receptors or both.

34. The method of claim 33 wherein the receptor includes the toll like receptor as receptor-4 protein (TLR-4).

35. The method of claim 34, prior to the step of delivering, further comprising a step of diagnosing the animal with an infectious condition in need of treatment where the infectious condition results from a pathogen that binds to the pattern recognition molecule.

36. The method of claim 23 wherein the co-active agent used in the step of delivering contains a treating agent targeting the pathogen.

37. The method of claim 36 wherein the treating agent for infectious disease includes an antibiotic.

38. The method of claim 37 wherein the antibiotic includes at least one member selected from the group consisting of aminoglycosides including amikacin, gentamicin, kanamycin, neomycin, netilmicin, streptomycin, and tobramycin; carbacephems including loracarbef, ertapenem, imipenem/cilastatin, and meropenem; cephalosporins including cefadroxil, cefazolin, cephalexin; cefaclor, cefamandole, cefoxitin, cefprozil, cefuroxime, cefixime, cefdinir, cefditoren, cefoperazone, cefotaxime, claforan, cefpodoxime, ceftazidime ceftibuten, ceftizoxime, ceftriaxone, cefepime, and maxipime; glycopeptides including teicoplanin and vancomycin; macrolides including azithromycin, clarithromycin, dirithromycin, eythromycin, and troleandomycin; monobactam inclosing aztreonam; penicillins inclosing amoxicillin, ampicillin, azlocillin, carbenicillin, cloxacillin, dicloxacillin, flucloxacillin, mezlocillin, nafcillin, penicillin, piperacillin, and ticarcillin; polypeptides including bacitracin, colistin, and polymyxin B; quinolones including ciprofloxacin, enoxacin, gatifloxacin, levofloxacin, lomefloxacin, moxifloxacin, norfloxacin, ofloxacin, and trovafloxacin; sulfonamides incluing mafenide, prontosil, sulfacetamide, sulfamethizole, sulfanilimide, sulfasalazine, sulfisoxazole, trimethoprim, and trimethoprim-sulfamethoxazole; tetracyclines including demeclocycline, doxycycline, minocycline, oxytetracycline, and tetracycline; and others including chloramphenicol, clindamycin, ethambutol, fosfomycin, furazolidone, isoniazid, linezolid, metronidazole, nitrofurantoin, pyrazinamide, quinupristin/dalfopristin, rifampin and spectinomycin.

39. The method of claim 20 wherein the co-active agent used in the step of delivering includes a nutrient that provides support for beneficial immune response.

40. The method of claim 39 wherein the nutrient includes at least one member selected from the group consisting of vitamins including vitamins A, B-6, biotin, C, D, and E.

41. The method of claim 40 wherein the nutrient includes at least one member selected from the group consisting of minerals including Cu, Fe, Se, Cr, Co, Zn, and salts thereof.

42. The method of claim 20 wherein the composition is formulated with pharmacologically compatible materials for oral administration, and the step of delivering is by oral administration.

43. The method of claim 20 wherein the composition is formulated with pharmacologically compatible materials for nasal administration and the step of delivering is by nasal administration.

44. The method of claim 20 wherein the composition is formulated with pharmacologically compatible materials for injectable administration and the step of delivering is by injectable administration.

45. The method of claim 20 wherein the composition is formulated with pharmacologically compatible materials for topical administration and the step of delivering is by topical administration.

46. The method of claim 20 wherein the composition is formulated as an animal feed and the step of delivering is by feeding the animal.

47. The method of claim 20 wherein the animal is a human animal.

48. The method of claim 20 wherein the animal is a food animal.

49. The method of claim 20 wherein the step of allowing the composition to work induces the maturation of the dendritic cells.

50. The method of claim 20 wherein the step of allowing the composition to work causes internalization of the receptor/carbohydrate complex and increases the rate and capture of an antigen or a mixture of antigens.

51. The method of claim 20 wherein the step of allowing the composition to work causes internalization of the receptor/carbohydrate complex and increases the rate and capture of an epitope or a mixture of epitopes.

52. The method of claim 20 wherein the step of allowing the composition to work causes internalization of the receptor/carbohydrate complex increasing the rate and capture of a hapten or a mixture of haptens.

53. The method of claim 20 wherein the step of allowing the composition to work causes internalization of the receptor/carbohydrate complex increasing the rate and capture of a hapten or a mixture comprised of antigens, epitopes and haptens.

54. The method of claim 20, prior to the step of delivering, further comprising a step of diagnosing the animal with a condition that is in need of treatment by use of the composition.

55. The method of claim 54 wherein the condition is an inflammatory disease.

56. The method of claim 54 wherein the condition includes an inflammatory component.

57. The method of claim 54 wherein the condition includes a suppressed immune system.

58. The method of claim 54 wherein the condition is caused by a pathogen.

59. The method of claim 54 wherein the condition is a cancer.

60. The method in claim 54 wherein the condition is an infection.

61. The method of claim 54 wherein the condition is a neurological disease.

62. The method in claim 54 wherein the condition is a cardiac disease.

63. The method of claim 54 wherein the condition is a blood disease.

64. The method of claim 54 wherein the condition is a skeletal disease.

65. The method of claim 54 wherein the condition is a disease of the muscle tissue.

66. The method of claim 54 wherein the condition is caused by a prion.

67. The method of claim 54 wherein the animal is a human.

68. The method of claim 54 wherein the animal is non-human.

69. The method of claim 54 wherein the co-active agent includes an antibiotic.

70. The method of claim 54 wherein the co-active agent includes an antifungal.

71. The method of claim 54 wherein the co-active agent includes an anti-viral.

72. The method of claim 54 wherein the co-active agent includes an anti-prion.

73. The method of claim 64 wherein the co-active agent includes humanized monoclonal antibodies.

74. The method of claim 54 wherein the co-active agent includes a humanized protein receptor with Fc immunoglobulin structure.

75. The method of claim 54 wherein the co-active agent includes an anti-inflammatory.

76. The method of claim 54 wherein the co-active agent includes a steroid.

77. The method of claim 54 further comprising a step of administering radiation ultraviolet or near visible therapy.

78. The method of claim 54 further comprising a step of administering radiation therapy.

79. The method of claim 54 further comprising a step of administering chemotherapy.

80. The method of claim 54 wherein the co-active agent includes at least one anti-cancer drug.

81. The method of claim 54 further comprising a step of administering at least two of the following: radiation therapy; chemotherapy; and an anticancer.

82. The method of claim 20 wherein the animal is a poultry species.

83. The method of claim 20 wherein the animal is an equine species.

84. The method of claim 20 wherein the animal is a bovine species.

85. The method of claim 20 wherein the animal is a primate species.

86. The method of claim 20 wherein the animal is a fish species.

87. The method of claim 20 wherein the composition used in the step of delivering is provided as a pelleted feed.

88. The method of claim 20 wherein the composition used in the step of delivering is provided as a confection.

89. The method of claim 20 wherein the composition used in the step of delivering is provided as a candy.

90. The method of claim 20 wherein the composition used in the step of delivering is provided as a bar, feed, or a snack.

91. The method of claim 20 wherein firstly there is an ex vivo treatment of DCs and later injection of these cells.

92. In a functional food material, the improvement comprising:
an effective amount of phosphorylated glucomannan polysaccharide to enhance immune function.

93. The functional food material of claim 92, wherein the phosphorylated glucomannan polysaccharide is isolated from Candida utilis.

94. The functional food material of claim 92, wherein the effective amount is present in a predetermined amount intended to provide from 0.1 mg to 1 mg per kg of body weight in a target animal that by is intended to consume the functional food material.

95. The functional food material of claim 92, wherein the functional food material is formulated as a food ingredient.

96. The functional food material of claim 95, wherein the food ingredient formulated as a flour additive.

97. The functional food material of claim 95, wherein the food ingredient formulated as a starch additive.

98. The functional food material of claim 95, wherein the food ingredient formulated as a spice additive.

99. The functional food material of claim 95, wherein the food ingredient formulated as a fat or oil additive.

100. The functional food material of claim 95, wherein the food ingredient formulated as protein.

101. The functional food material of claim 95, wherein the food ingredient is formulated as a carbohydrate additive.

102. The functional food material of claim 95, wherein the food ingredient is formulated as a vitamin additive.

103. The functional food material of claim 95, wherein the food ingredient is formulated as a sugar additive.

104. The functional food material of claim 95, wherein the food ingredient is formulated as a mineral additive.

105. The functional food material of claim 95, wherein the food ingredient is formulated as a thickener.

106. The functional food material of claim 95, wherein the food ingredient is formulated as a thickener.

107. The functional food material of claim 92 formulated as a beverage.

108. The functional food material of claim 92 formulated as a cereal bar.

109. The functional food material of claim 92 formulated as a dessert.

110. The functional food material of claim 92 formulated as a baked good.

111. The functional food material of claim 92 essentially free of storage protein from nongerminated seeds of Ricinus communis.

112. In a cosmetic material, the improvement comprising:
an effective amount of phosphorylated glucomannan polysaccharide to enhance immune function.

113. The cosmetic material of claim 111 formulated as a product selected from the group consisting of lipstick, cosmetic makeup, fingernail polish, eyeliner, and phosphorylated glucomannan polysaccharide mixed with an ingredient for making these products.

114. The cosmetic material of claim 111 formulated as a product selected from the group consisting of eye drops, ear drops, antibacterial ointment or liquids, deodorant, burn cream, haemorrhoid ointment, analgesic ointment or solution, athlete's foot creams or powders, veterinary ointments, medicaments, suntan lotion, wax or chemicals preparations for the removal of hair, insect repellent, and phosphorylated glucomannan polysaccharide mixed with an ingredient for making these products.

115. The cosmetic material of claim 111 formulated as a product selected from the group consisting of deodorant, face cream, soap, skin cleanser, skin care preparation, cosmetic gel, moisturizers, shampoo, perfume, conditioner, medicaments, and phosphorylated glucomannan polysaccharide mixed with an ingredient for making these products.

116. The cosmetic material of claim 111, wherein the cosmetic material contains at least one ingredient selected from the group consisting of pigment, emollient, thickener, preservative, vitamin, bactericide, fungicide, humectant, gel, pH adjusting agent, collagen, aldehyde, herbal supplement, botanical extract, alcohol, petrolatum, surfactant, and fragrance.

117. The cosmetic material of claim 111, wherein the cosmetic material is essentially free of essentially free of storage protein from nongerminated seeds of Ricinus communis.

118. In a dosage form made ready for consumption to deliver phosphorylated glucomannan polysaccharide in an effective amount to enhance immune function, the improvement comprising the dosage form being essentially free of essentially free of storage protein from nongerminated seeds of Ricinus communis.


SEQUENCE LISTING (1 sequence)

SEQ ID NO: 1 - protein, 404 aa, Homo sapiens
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA